(19) United States
(12) Patent Application Publication
(10) Pub. No.: US 2024/0173324 A1
(43) Pub. Date: May 30, 2024

(54) COMBINATORY THERAPY FOR PREVENTING, INHIBITING, TREATING, OR REDUCING ANEURYSMS

(71) Applicant: The Regents of the University of California, Oakland, CA (US)

(72) Inventor: Hua Cai, Los Angeles, CA (US)

(21) Appl. No.: 18/280,074

(22) PCT Filed: Mar. 2, 2022
(86) PCT No.: PCT/US22/18501

§ 371 Date: Sep. 1, 2023

Related U.S. Application Data
(60) Provisional application No. 63/155,348, filed on Mar. 2, 2021.

(51) Int. Cl. A61K 31/519; A61K 31/4422; A61P 9/00; A61P 39/06

(52) U.S. Cl. CPC A61K 31/519; A61K 31/4422; A61P 9/00; A61P 39/06

(57) ABSTRACT

The present disclosure relates to pharmaceutical compositions comprising a folate compound and a calcium channel blocker, as well as the method for using such pharmaceutical compositions in the treatment of aneurysms.

### RELATED APPLICATIONS

This application claims the benefit of U.S. Provisional Application No. 63/155,348, filed Mar. 2, 2021, and U.S. Provisional Application No. 63/170,774, filed Apr. 5, 2021, each of which is hereby incorporated by reference in its entirety.

### GOVERNMENT SUPPORT CLAUSE

This invention was made with government support under Grant Number HL077440, awarded by the National Institutes of Health. The government has certain rights in the invention.

### BACKGROUND

Aortic aneurysms are prevalent and severe vascular diseases, with high mortality resulting from patients dying of unpredictable sudden rupture of the aneurysms. No treatment options have been available except for surgical correction of large aneurysms with considerable risk. Abdominal aortic aneurysm (AAA) is defined as an abdominal aortic dilation of over 3 cm in diameter, most commonly affecting the infrarenal segment. It is associated with high risk of mortality in the event of aneurysm rupture, leading to around 200,000 deaths each year worldwide. The incidence of AAA is up to 9% in population older than 65, and a meta-analysis of 56 studies indicates that the prevalence of AAA in general population is at 4.8%. While the most well-recognized risk factors for AAA include male gender and smoking, other risk factors have been implicated in AAA formation such as older age, family history, hypertension and hyperlipidemia. The mechanisms of AAA formation are complex, primarily involving oxidative stress driven vascular remodeling that is characterized by matrix degradation and inflammation, resulting in expansion of abdominal aortas. The only clinical intervention to treat aortic aneurysms is limited to surgical correction of large AAAs of over 5.5 cm in size, and the 30-day mortality rate is high for both endovascular aortic aneurysm repair (EVAR) and open surgical repair at 1.7-4.7%. There have been no oral medicines available to treat all sizes of the aortic aneurysms including the smaller and growing aneurysms to prevent unpredictable sudden rupture and death.

Thoracic aortic aneurysm is a term used to describe aneurysms formed at the thoracic region of the aorta, including ascending thoracic aorta/aortic root area (about 60%), and descending thoracic aorta area (about 40%). Thoracic aortic aneurysms may be caused by arteriosclerosis (atherosclerosis), increased blood pressure (hypertension), congenital diseases such as Marfan Syndrome or trauma. The prevalence of TAA is around 4.2% in general population. Likewise to AAA, no treatment options/oral medications have been available except for surgical correction. Oxidative stress-driven vascular remodeling is a common pathway shared between AAA and TAA for aneurysm formation.

Cerebral aneurysms develop as a result of thinned/weakened artery walls. Aneurysms often form at forks or branches in arteries because those sections of the vessel are weaker and sensitive to vascular remodeling to result in aneurysm formation. Cerebral aneurysms are one of the most common cerebrovascular diseases. If the aneurysm is severe, it can develop into a hemorrhagic stroke with cerebrovascular bleeding. Likewise to AAA/TAA, no treatment options/oral medications have been available except for surgical correction. Oxidative stress-driven vascular remodeling is a common pathway shared between AAA/TAA and cerebral aneurysm for aneurysm formation.

### SUMMARY OF THE INVENTION

Provided herein are compositions, kits and methods for preventing or treating an abdominal aortic aneurysm (AAA), a thoracic aortic aneurysm (TAA), or a cerebral aneurysm. In certain aspects, provided herein are pharmaceutical compositions comprising a folate compound and a calcium channel blocker. Also provided herein are kits comprising a folate compound and a calcium channel blocker.

The methods provided herein comprise preventing or treating aneurysms (e.g., an abdominal aortic aneurysm (AAA), a thoracic aortic aneurysm (TAA), or a cerebral aneurysm) by conjointly administering a folate compound and a calcium channel blocker to a subject in need thereof. In some embodiments, the methods comprise ameliorating at least one symptom of an aneurysm or a symptom associated with an aneurysm by conjointly administering a folate compound and a calcium channel blocker to a subject in need thereof. The symptom may be, for example, increased superoxide production, increased eNOS uncoupling activity, decreased nitric oxide (NO) bioavailability, decreased tetrahydrobiopterin (H4B) bioavailability, enlargement of blood vessels (abdominal aortas, thoracic aortas or blood vessels in the brain), increased vascular remodeling, increased elastin degradation (flattening and breakdown), increased vascular inflammation/macrophage infiltration, increased matrix metalloproteinase (MMP) activation, increased adventitial hypertrophy, or a decrease in eNOS function.

Also provided herein are methods of decreasing superoxide production, eNOS uncoupling activity, enlargement of blood vessels (abdominal aortas, thoracic aortas or blood vessels in the brain), vascular remodeling, elastin degradation (flattening and breakdown), vascular inflammation/macrophage infiltration, matrix metalloproteinase (MMP) activation, and/or adventitial hypertrophy in a subject afflicted with an aneurysm, comprising conjointly administering a folate compound and a calcium channel blocker to a subject in need thereof. In some embodiments, provided herein are methods of increasing eNOS function, nitric oxide (NO) and/or tetrahydrobiopterin bioavailabilities in a subject afflicted with an aneurysm by conjointly administering a folate compound and a calcium channel blocker to a subject in need thereof.

An aneurysm described herein may be an AAA aneurysm, a TAA aneurysm, or a cerebral aneurysm.

The folate compound may be represented by formula I

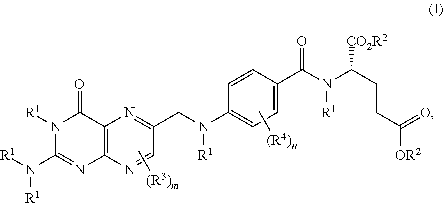

or a pharmaceutically acceptable salt thereof, wherein:
each R¹ independently is hydrogen, acyl, ester, amide, alkyl, alkenyl, alkynyl, carbocyclyl, heterocyclyl, aryl, or heteroaryl;
each R² independently is hydrogen, alkyl, alkenyl, alkynyl, carbocyclyl, heterocyclyl, aryl, or heteroaryl;
each R³ and R⁴ independently is halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl;
m is an integer selected from 0-3; and
n is an integer selected from 0-4.

Each R¹ independently is hydrogen, acyl, ester, amide, or alkyl. In some embodiments, each R¹ is hydrogen. Each R² independently may be hydrogen or alkyl. In some embodiments, each R² is hydrogen. M and/or n may be 0.

In some embodiments, the folate compound is

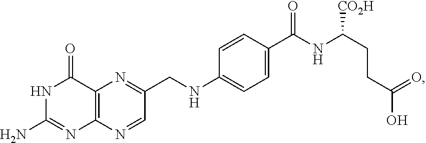 [SMILES: Nc1nc2ncc(CNc3ccc(C(=O)N[C@@H](CCC(=O)O)C(=O)O)cc3)nc2c(=O)[nH]1]

or a pharmaceutically acceptable salt thereof.

The calcium channel blocker may be a dihydropyridine compound. For example, wherein the dihydropyridine compound is represented by formula II:

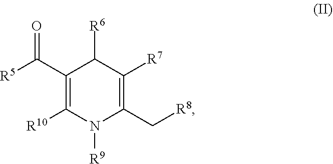

or a pharmaceutically acceptable salt thereof, wherein:
R⁵, R⁶, R⁷, R⁸, and R¹⁰ each independently is hydrogen, halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl; and
R 9 is hydrogen, acyl, ester, amide, alkyl, alkenyl, alkynyl, carbocyclyl, heterocyclyl, aryl, or heteroaryl.

The R⁶ may be alkyl, alkenyl, alkynyl, carbocyclyl, heterocyclyl, aryl, or heteroaryl. In some embodiments, R⁶ is methyl or substituted or unsubstituted phenyl. R⁶ may be a phenyl, optionally substituted with halogen, haloalkyl, alkyl, or nitro.

The dihydropyridine compound may be represented by formula II-a, II-b, II-c, or II-d:

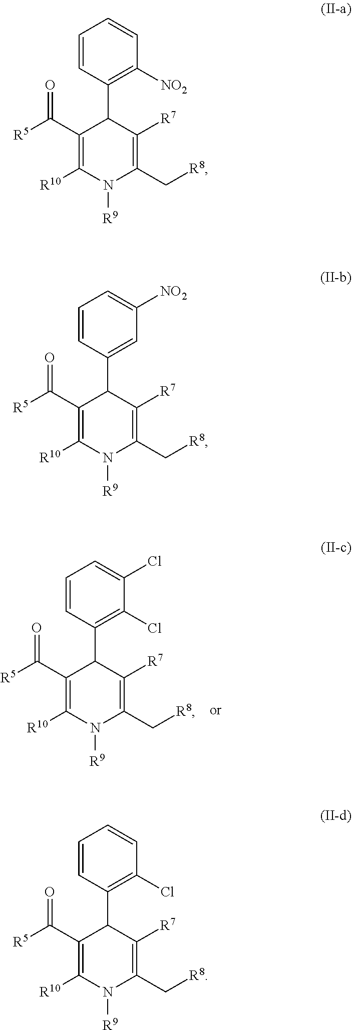

R⁹ may be a hydrogen, alkyl, alkenyl, alkynyl, carbocyclyl, heterocyclyl, aryl, or heteroaryl. In some embodiments, R⁹ is an alkyl optionally substituted with halogen, amino, hydroxyl, alkoxy, cyano, nitro, acyl, ester, amide, alkylthio, carbocyclyl, heterocyclyl, aryl, or heteroaryl.

In some embodiments, R⁹ is

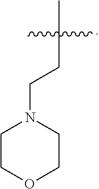 [SMILES: *CCN1CCOCC1]

In some embodiments, R¹⁰ is cyano, amino, alkyl, alkenyl, alkynyl, carbocyclyl, heterocyclyl, aryl, or heteroaryl. R¹⁰ may be methyl.

In some embodiments, the dihydropyridine compound is represented by formula II-a-1, II-b-1, II-c-1, or II-d-1:

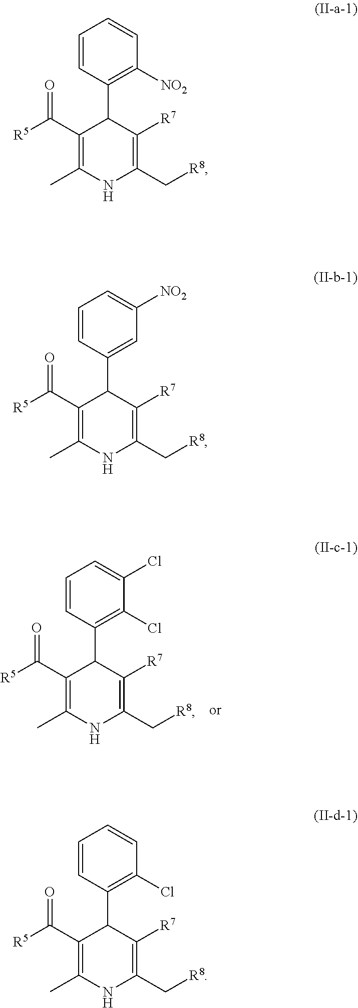

R⁵ may be an alkoxy, amino, alkyl, carbocyclyl, heterocyclyl, aryl, or heteroaryl. In some embodiments, R⁵ is alkoxy optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl.

In some embodiments, R⁵ is

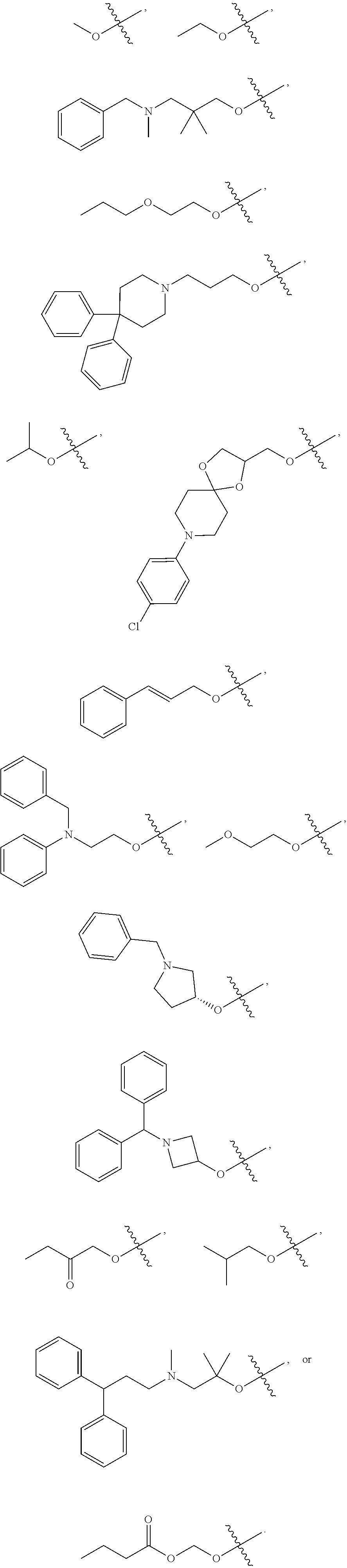

R⁷ may be an acyl, ester, amide, alkyl, alkenyl, alkynyl, carbocyclyl, heterocyclyl, aryl, or heteroaryl. In some embodiments, R⁷ is

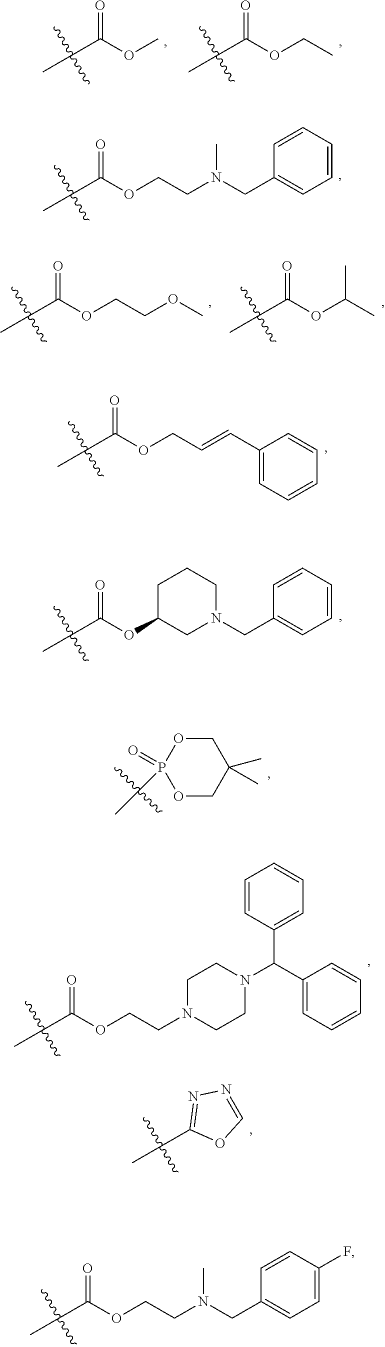

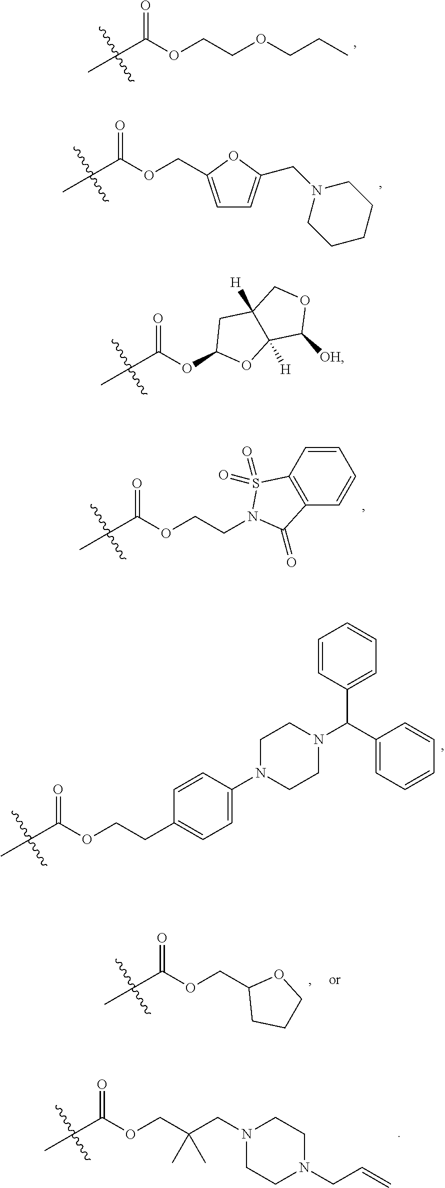

The R⁸ may be a hydrogen, hydroxyl, alkoxy, alkylthio, alkyl, alkenyl, alkynyl, carbocyclyl, heterocyclyl, aryl, or heteroaryl. R⁸ may be a hydrogen, hydroxyl, alkoxy, alkylthio, or alkyl optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl. In some embodiments, R⁸ is hydrogen,

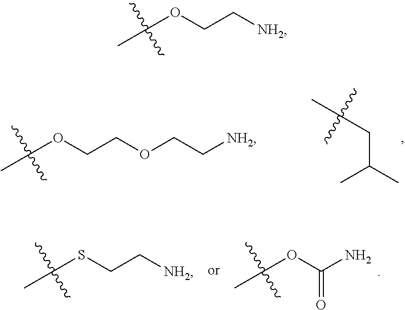

In some embodiments, the dihydropyridine compound is selected from:

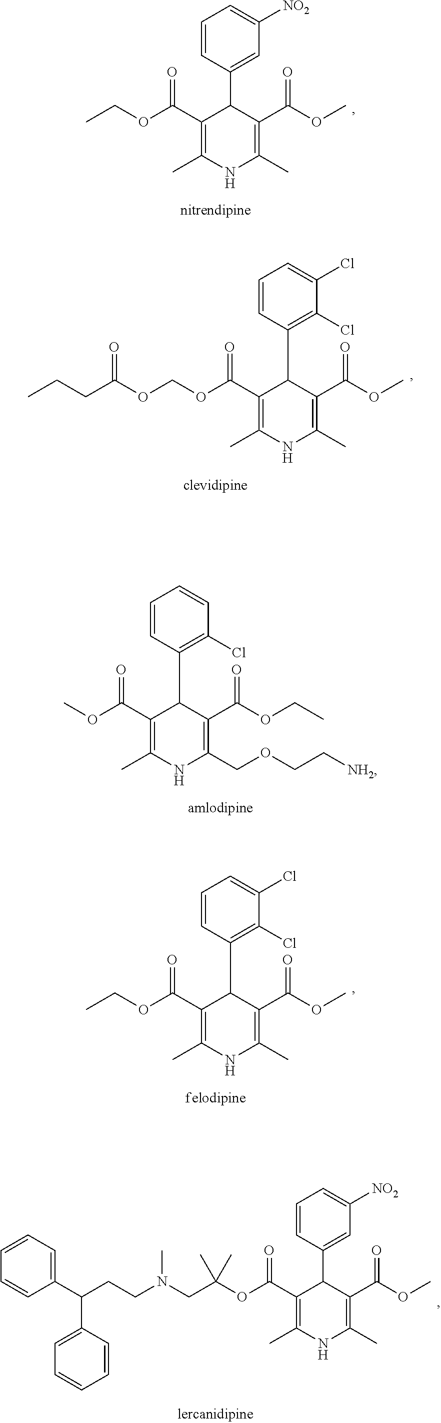

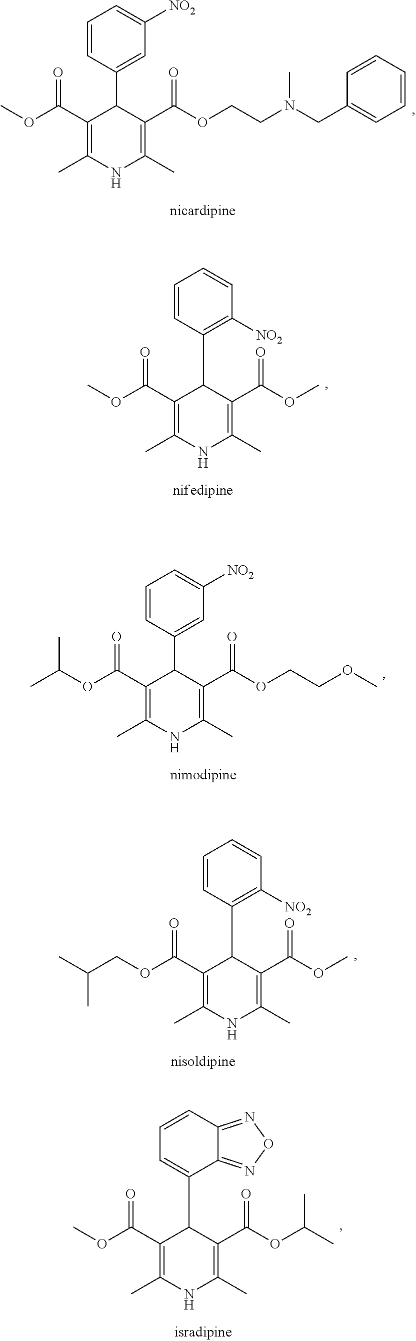

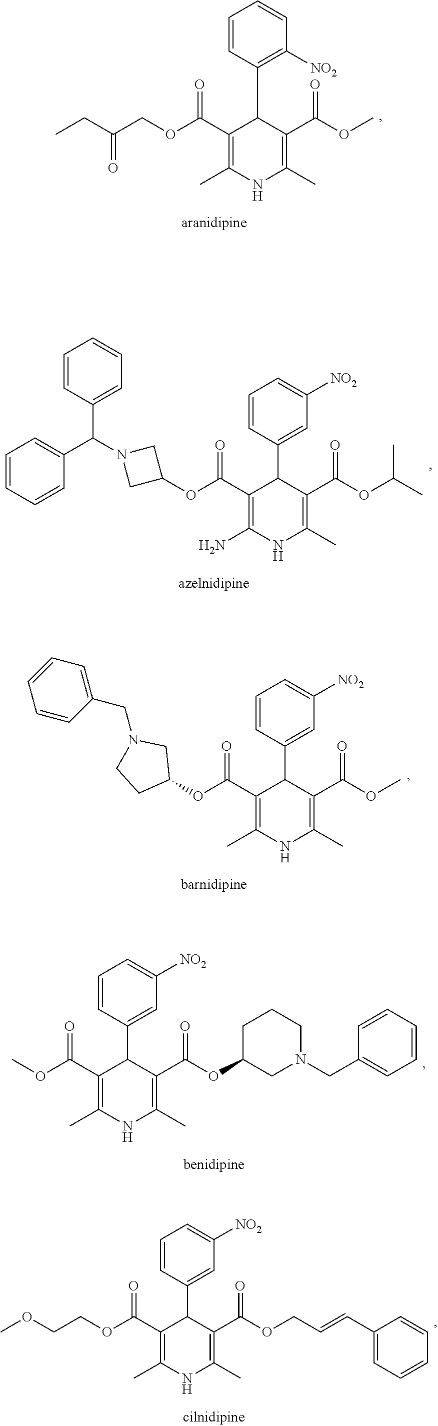

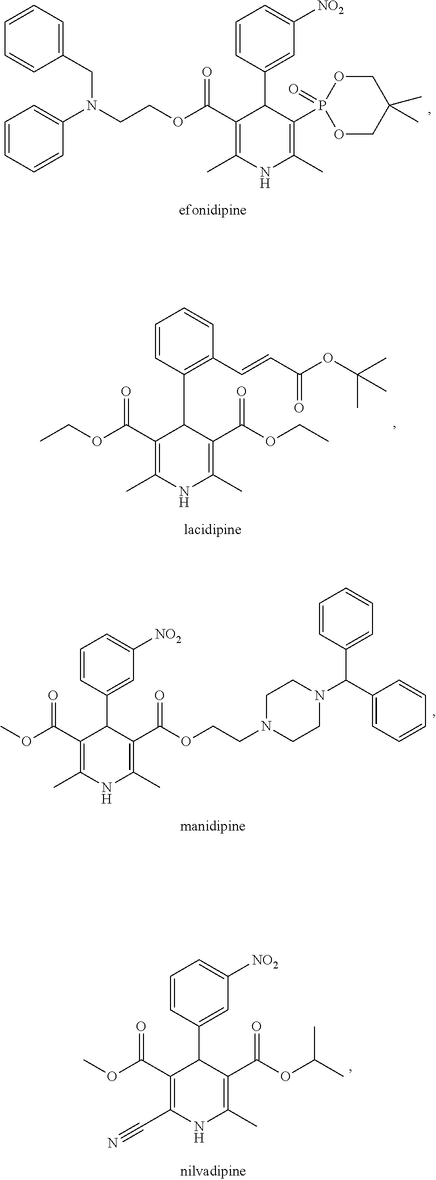

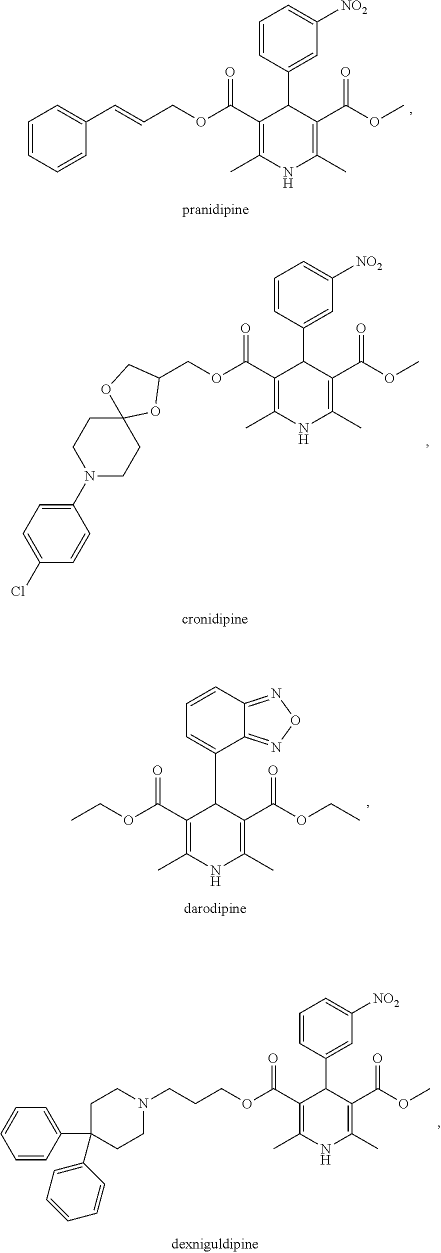

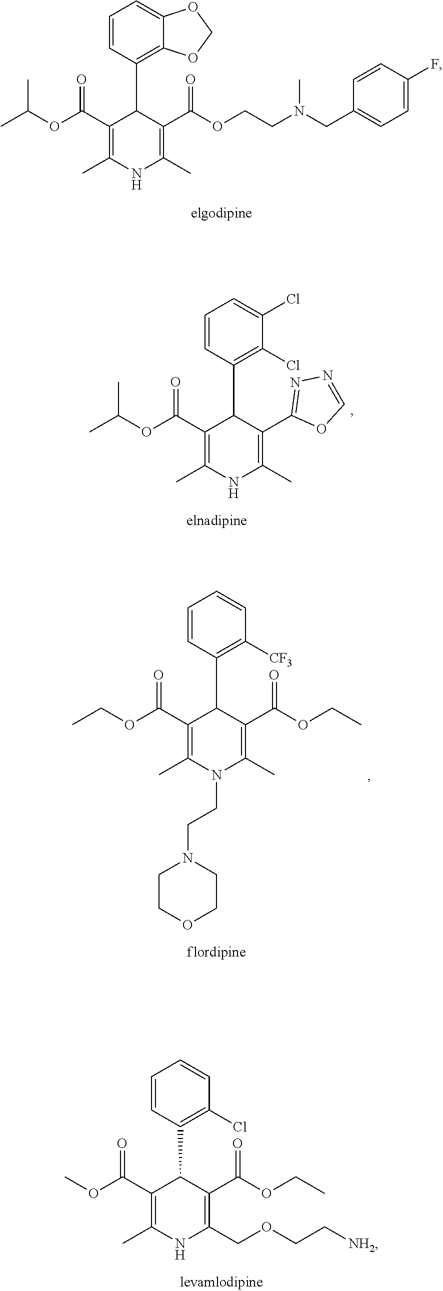

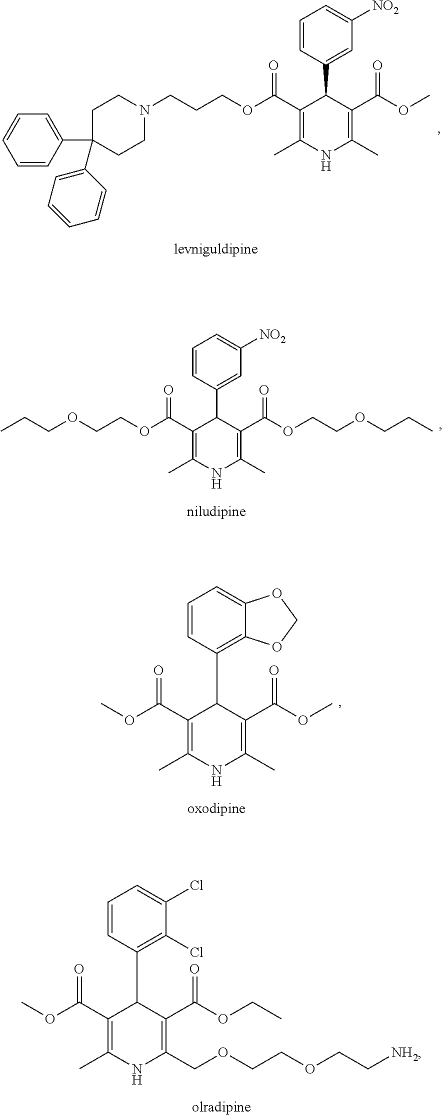

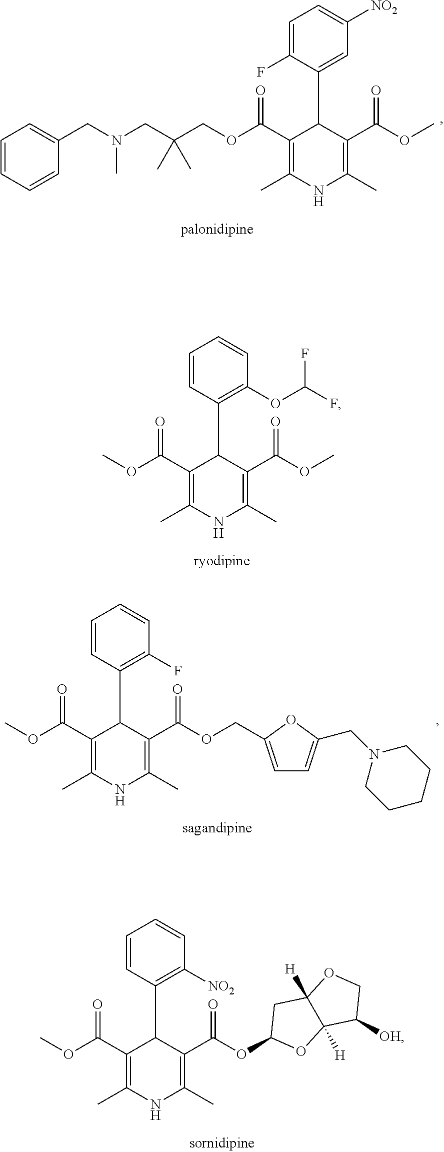

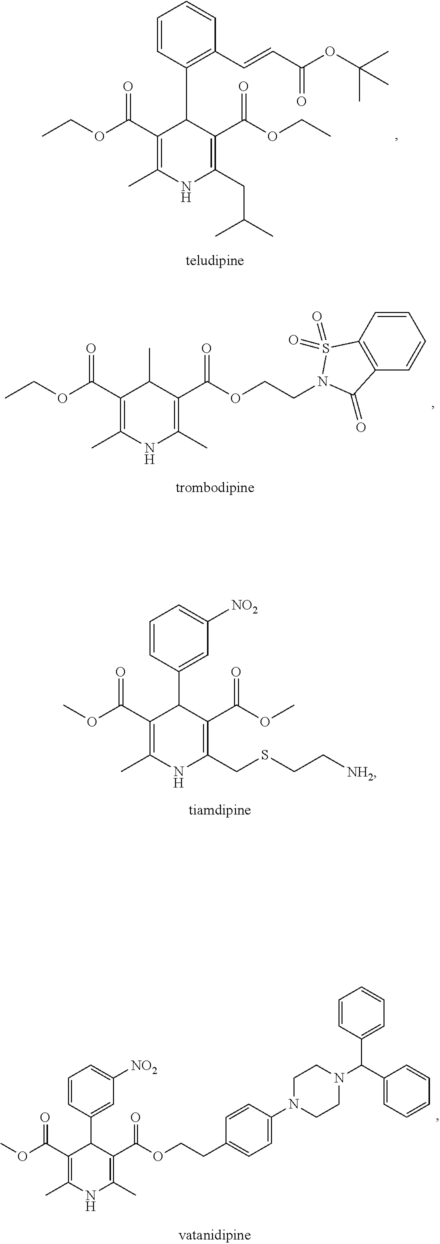

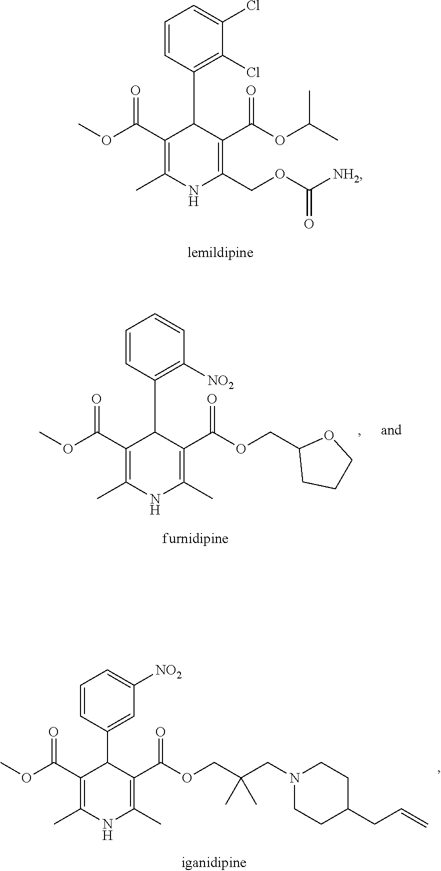 [SMILES: COC(=O)C1=C(C)NC(COC(N)=O)=C(C(=O)OC(C)C)C1c1cccc(Cl)c1Cl ; COC(=O)C1=C(C)NC(C)=C(C(=O)OCC2CCCO2)C1c1ccccc1[N+](=O)[O-] ; C=CCC1CCN(CC(C)(C)COC(=O)C2=C(C)NC(C)=C(C(=O)OC)C2c2cccc([N+](=O)[O-])c2)CC1]

or a pharmaceutically acceptable salt thereof.

In some embodiments, the dihydropyridine compound is

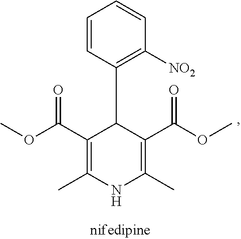

or a pharmaceutically acceptable salt thereof.

The folate compound and the dihydropyridine compound may be in the same composition. The folate compound may be folic acid. The calcium channel blocker may be Nifedipine. In some embodiments, the amount administered of the folate compound and the amount administered of the calcium channel blocker are therapeutically effective. In some embodiments, the folate compound and the calcium channel blocker are administered in a mass ratio of about 10:1 to about 1:10.

The folate compound and the calcium channel blocker may be administered in a mass ratio of about 3:1 to about 3:4. The folate compound may be administered in an amount of about 1-350 mg, about 1-700 mg, about 1-1050 mg, about 1-1400 mg, or about 1-1750 mg. In some embodiments, the calcium channel block is administered in an amount of about 1-350 mg, about 1-700 mg, about 1-1050 mg, about 1-1400 mg, about 1-1750 mg, about 1-2100 mg, or about 1-2450 mg. The folate compound and the calcium channel blocker may be administered simultaneously or sequentially. In some embodiments, the folate compound and the calcium channel blocker are in separate dosage forms.

The aneurysm may be an abdominal aortic aneurysm, cerebral aneurysm, or thoracic aortic aneurysm. The folate compound and the calcium channel blocker may be administered orally.

### BRIEF DESCRIPTION OF THE DRAWINGS

FIG. 1A-FIG. 1B shows a combination of FA with Nifedipine substantially and completely further improved efficacy of FA in attenuating incidence of aortic aneurysm in a dose-dependent manner. ApoE null mice of 6-8 months old were infused with angiotensin II (Ang II, 1000 ng/mg/min) for 4 weeks in the presence of regular chew, customized chew containing folic acid (FA, 15 mg/kg/day), or customized chew containing FA and various doses of Nifedipine (1.5, 5.0, and 20 mg/kg/day). FIG. 1A shows the incidence of abdominal aortic aneurysm (AAA) in cross different groups. The data indicate that with regular chow, the AAA incidence in Ang II infused apoE mice is 85.71%, while it was attenuated by oral administration of FA to 18.75%. Combination of FA with 1.5, 5 or 20 mg/kg/day Nifedipine substantially and completely further attenuated AAA incidence rate to 12.50%, 11.76% and 0.00% respectively. *p<0.05, ***p<0.001, n=17-35. FIG. 1B shows actual numbers of animals examined across different groups and numbers of animals developed AAA: apoE, n=34/0; apoE+Ang II, n=35/30; apoE+Ang II+FA, n=32/6; apoE+Ang II+FA+Nif (1.5), n=24/3; apoE+Ang II+FA+Nif (5.0), n=17/2;, apoE+Ang II+FA+Nif (20.0), n=18/0.

FIG. 2A-FIG. 2B shows that combination of FA with Nifedipine substantially and completely further attenuated enlargement of abdominal aortas in a dose-dependent manner. ApoE null mice of 6-8 months old were infused with angiotensin II (Ang II, 1000 ng/mg/min) for 4 weeks in the presence of regular chew, customized chew containing folic acid (FA, 15 mg/kg/day), or customized chew containing FA and various doses of Nifedipine (1.5, 5.0, and 20 mg/kg/day). Enlargement of abdominal aortas was monitored weekly using ultrasound. FIG. 2A shows representative weekly ultrasound images taken from different experimental groups. FIG. 2B shows grouped data of ultrasound measurements of abdominal aortic areas. Infusion of Ang II into apo E null mice resulted in marked enlargement in abdominal aortas (***p<0.001 vs. apoE of the same week). Oral administration with FA alone significantly attenuated enlargement of abdominal aortas (##p<0.01 or ###p<0.001 vs. apoE+Ang II of the same week). Compared to the FA alone group, Nifedipine at doses of 1.5, 5.0 and 20 mg/kg/day substantially/completely further attenuated enlargement of abdominal aortas in a dose-dependent manner (@@p<0.01 or @@@p<0.001 for 1.5 mg/kg/day Nifedipine group; &&p<0.01 or &&&p<0.001 for 5.0 mg/kg/day Nifedipine group; $$$p<0.001 for 20 mg/kg/day Nifedipine group). Of note, at some time points, Nifedipine 5.0 or 20 mg/kg/day group was significantly more effective than Nifedipine 1.5 mg/kg/day group (¥p<0.05; %p<0.05 or %%% p<0.001). n =4-19.

FIG. 3 shows combination of FA with Nifedipine substantially and completely further attenuated vascular remodeling in a dose-dependent manner. ApoE null mice of 6-8 months old were infused with angiotensin II (Ang II, 1000 ng/mg/min) for 4 weeks in the presence of regular chew, customized chew containing folic acid (FA, 15 mg/kg/day), or customized chew containing FA and various doses of Nifedipine (1.5, 5.0, and 20 mg/kg/day). Data of H&E staining indicate that vascular remodeling characterized by elastic degradation and adventitial hypertrophy in Ang II infused apoE null mice was significantly attenuated by oral administration of FA, which was substantially and completely further alleviated by combination of FA with various doses of Nifedipine in a dose-dependent manner.

FIG. 4 shows combination of FA with Nifedipine substantially and completely further attenuated elastin degradation in a dose-dependent manner. ApoE null mice of 6-8 months old were infused with angiotensin II (Ang II, 1000 ng/mg/min) for 4 weeks in the presence of regular chew, customized chew containing folic acid (FA, 15 mg/kg/day), or customized chew containing FA and various doses of Nifedipine (1.5, 5.0, and 20 mg/kg/day). Data of VVG staining indicate that medial elastin degradation featured by elastin flattening and breakdown in Ang II infused apoE null mice was significantly attenuated by oral administration of FA, which was substantially and completely further alleviated by combination of FA with various doses of Nifedipine in a dose-dependent manner.

FIG. 5A-FIG. 5B shows that combination of FA and Nifedipine substantially and completely further attenuated total superoxide production and eNOS uncoupling in a dose-dependent manner. ApoE null mice of 6-8 months old were infused with angiotensin II (Ang II, 1000 ng/mg/min) for 4 weeks in the presence of regular chew, or oral administration of folic acid (FA, 15 mg/kg/day) in combination with various doses of Nifedipine (1.5, 5.0, and 20 mg/kg/day). FIG. 5A shows total superoxide production determined by electron spin resonance (ESR). The data indicate that there was a significant increase in total superoxide production in aortas isolated from Ang II infused apoE null mice, which was markedly abrogated by oral FA administration. Combination of FA with 1.5, 5 and 20 mg/kg/day of Nifedipine substantially and completely further attenuated total superoxide production in a dose-dependent manner. **p<0.01, ***p<0.001, n =5-7. FIG. 5B shows aortic eNOS uncoupling activity determined by ESR. The data indicate that eNOS uncoupling activity, reflected by L-NAME-inhibitable superoxide production, was completely attenuated by oral administration of FA alone or in combination with various dosing of Nifedipine. *p<0.05, ** p<0.01, n =5-7. A reduction in superoxide production with L-NAME indicates that eNOS is uncoupled producing superoxide, while an increase in superoxide production with L-NAME indicates that eNOS is coupled producing NO.

FIG. 6 shows a combination of FA and Nifedipine substantially further improved NO bioavailability in a dose-dependent manner. ApoE null mice were infused with Ang II, fed regular chow or chow mixed with FA (15 mg/ kg/day) or chow mixed with FA plus different concentration of Nifedipine (1.5, 5 and 20 mg/kg/day). ApoE null mice of 6-8 months old were infused with angiotensin II (Ang II, 1000 ng/mg/min) for 4 weeks in the presence of regular chew, or oral administration of folic acid (FA, 15 mg/kg/day) in combination with various doses of Nifedipine (1.5, 5.0, and 20 mg/kg/day). Aortic NO bioavailability was determined by electron spin resonance (ESR). The data indicate that there was a significant decrease in NO bioavailability in aortas isolated from Ang II infused apoE null mice, which was significantly restored by oral FA administration. Combination of FA with 1.5, 5 and 20 mg/kg/day of Nifedipine substantially and dose-dependently further restored NO bioavailability. *p<0.05, **p<0.01, ***p<0.001, n =6-8.

FIG. 7 shows a combination of FA and Nifedipine substantially further improved aortic H4B bioavailability in a dose-dependent manner. ApoE null mice were infused with Ang II, fed regular chow or chow mixed with FA (15 mg/ kg/day) or chow mixed with FA plus different concentration of Nifedipine (1.5, 5 and 20 mg/kg/day). Aortic H4B bioavailability was determined by HPLC. The data indicate that there was a significant decrease in aortic H4B levels in Ang II infused apoE null mice, which was markedly restored by oral FA administration. Combination of FA with 1.5, 5 and 20 mg/kg/day of Nifedipine substantially and dose-dependently further restored H4B bioavailability. *p<0.05, **p<0.01, *** <0 001 n =4-10.

### DETAILED DESCRIPTION

Oxidative stress plays an important role in the development of AAA. A novel role of endothelial nitric oxide synthase (eNOS) has been previously established in AAA formation via sustaining oxidative stress to induce MMP activity and matrix degradation. It has been demonstrated that eNOS uncoupling mediates AAA formation in a novel model of angiotensin II (Ang II) infused hph-1 mice, in which 79% of the mice developed AAA within 2 weeks of Ang II infusion, with 14% died of ruptured aneurysm (Gao L et al., Hypertension (2012)). This is the most robust AAA model to date. Restoration of dihydrofolate reductase (DHFR) expression with oral folic acid (FA) administration to recouple eNOS markedly attenuated AAA formation in Ang II infused hph-1 mice. Moreover, a novel role of eNOS uncoupling in the development of AAA in Ang II-infused apoE null mice has been observed, the well-established, classical model of AAA, where oral FA administration also effectively restored DHFR expression to recouple eNOS, resulting in abrogated aneurysm formation. In addition, the inventors have further elucidated a novel role of eNOS uncoupling in mediating thoracic aortic aneurysm (TAA) and AAA formation in Fbnic1^{C1039G/+} Marfan Syndrome (MFS) mice, targeting of which with FA diet recouples eNOS to reduce superoxide production and restore NO bioavailability, resulting in attenuated formation of TAA and AAA. Therefore, these data further confirm a significant role of uncoupled eNOS in mediating AAA and TAA formation, and demonstrate a universal efficacy of FA in attenuating AAA and TAA formation via improvement in DHFR expression to restore eNOS coupling activity.

Hypertension is a risk factor for AAA, and AAA patients often have co-existing hypertension. An earlier study also investigated effects on AAA formation of both low and high doses of the anti-hypertensive drug Nifedipine, which is a calcium channel blocker. While low dose of Nifedipine has no effects on blood pressure, both low and high dose of Nifedipine were able to attenuate AAA formation, via restoration of DHFR expression and inhibition of NADPH oxidase to prevent eNOS uncoupling. The high dose of Nifedipine can also reduce blood pressure at the same time, hence particularly valuable to treat patients with both AAA and co-existing hypertension. Nonetheless, neither FA alone nor Nifedipine alone is sufficient to fully attenuate AAA formation. Hence, the goal of the present study is to examine whether combinatory therapy of FA and Nifedipine has synergistic effects in the suppression of AAA growth with augmented efficacy. Ang II-infused apoE null mice were therefore treated with combinatory therapy of FA and various doses of Nifedipine (1.5, 5 or 20 mg//kg/day), which exhibited novel and substantially improved therapeutic effects on AAA formation in a dose-dependent manner. The combination of FA with various doses of Nifedipine was more effective in substantially and completely further attenuating total superoxide production and eNOS uncoupling activity in a dose-dependent manner, resulting in substantially and completely attenuated vascular remodeling to alleviate formation of aneurysms; with the FA and high dose of Nifedipine (20 mg/kg/day) completely inhibiting formation of AAA to zero occurrence. This was accompanied by dose-dependently further improved NO bioavailability and H4B bioavailability, going along with restoration in eNOS function/coupling activity. The enlargement of abdominal aortas defined by ultrasound was substantially and completely further abrogated by combination of FA with various doses of Nifedipine in a dose-dependent manner as well, with FA plus high dose of Nifedipine (20 mg/kg/day) attenuating sizes of abdominal aortas 100% to control levels. These findings are remarkable in establishing that novel combinatory therapy of FA and Nifedipine can serve as a robust, first-in-case, and most effective oral medicine for the treatment of aortic aneurysms.

The present invention is based, in part, on the discovery that combinatory therapy of FA and Nifedipine alleviates vascular remodeling featured by elastin degradation and adventitial hypertrophy in a dose-dependent manner in a model of AAA. The protective effects on AAA incidence and related pathophysiological changes are attributed to augmented effects of combinatory therapy on restoration of eNOS function and abrogation of oxidative stress. Combination of FA with various doses of Nifedipine substantially and completely further attenuated superoxide production in a dose-dependent manner while completely recoupling eNOS to restore NO bioavailability also in a dose-dependent manner. The aortic bioavailability of eNOS cofactor H₄B was indeed further improved by the combinatory therapy, going along with the outcome of eNOS recoupling. A decrease in eNOS uncoupling may be quantified as an increase in eNOS cofactor H₄B. These data demonstrate that combinatory therapy of FA and Nifedipine can serve as a novel oral medication that is effective in the treatment of aortic aneurysms. The role of eNOS uncoupling and oxidative stress in mediating aneurysm formation is a common pathway for AAA, cerebral aneurysm, and thoracic aortic aneurysm (TAA) (Gao et al. Hypertension (2012), Huang et al. Redox Biology (2021) and Starke et al. Curr Neurovasc Res. 2013 August; 10(3): 247-255). Therefore, the combination treatment disclosed herein can be used to prevent or treat AAA, cerebral aneurysm and/or TAA. Given the common pathway between both TAA, cerebral aneurysm and AAA, the combination treatments disclosed herein would show synergy when administered to a subject afflicted with TAA, cerebral aneurysm and/or AAA.

In one aspect, provide herein are pharmaceutical compositions comprising a folate compound and a calcium channel blocker.

### Pharmaceutical Compositions

The compositions, kits, and methods of the present invention may be utilized to treat an individual in need thereof (e.g., an individual suffering from an AAA, cerebral or TAA aneurysm). In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier.

The one or more folic acid compounds and one or more calcium channel blockers (e.g., Nifedipine compounds) may be administered, preferably in the form of pharmaceutical compositions, to a subject, in therapeutically effective amounts. In some embodiments, a therapeutically effective amount of the one or more folic acid compounds and one or more calcium channel blockers (e.g., Nifedipine compounds) ranges from about 0.01-60 mg/kg body weight, about 0.01-45 mg/kg body weight, about 0.01-30 mg/kg body weight, or about 0.01-15 mg/kg body weight of the one or more folic acid compounds, and about 0.01-40 mg/kg body weight, about 0.01-35 mg/kg body weight, about 0.01-30 mg/kg body weight, about 0.01-25 mg/kg, about 0.01-20 mg/kg, about 0.01-15 mg/kg, about 0.01-10 mg/kg, about 0.01-5 mg/kg, or about 0.01-1.5 mg/kg body weight of the one or more calcium channel blockers (e.g., Nifedipine compounds). In some embodiments, the ratio of the one or more folic acid compounds to the one or more calcium channel blockers (e.g., Nifedipine compounds) that are administered ranges from about 10:1 to about 1:10. In some embodiments, the amount of the one or more folic acid compounds and one or more calcium channel blockers (e.g., Nifedipine compounds) are provided or administered in a synergistic amount or a synergistic ratio. In some embodiments, the amount of the one or more folic acid compounds administered to the subject is about 1-350 mg, about 1— 700 mg, about 1-1050 mg, about 1-1400 mg, or about 1-1750 mg, and the amount of the one or more calcium channel blockers (e.g., Nifedipine compounds) administered to the subject is about 1-350 mg, about 1-700 mg, about 1-1050 mg, about 1-1400 mg, about 1-1750 mg, about 1-2100 mg, or about 1-2450 mg. The dosages used for treatment may increase or decrease over the course of a given treatment.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans; antioxidants, such as ascorbic acid or glutathione; chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro-emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for the same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.01 percent to about 99.99 percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Any one of the compounds or agents disclosed herein may be administered parenterally.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.01 to 99.99% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow-release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents. Any one of the compounds or agents disclosed herein may be administered conjointly.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation. As used herein, a “therapeutically effective amount” or a “therapeutically effective dose” includes, but is not limited to, amounts or doses that show synergy or technical effect when administered (e.g., conjointly administered) with another compound or agent disclosed herein.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

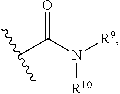

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

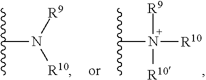

wherein R⁹, R¹⁰, and R¹⁰ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

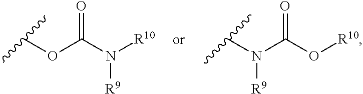

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—. The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O-. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a =0 substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

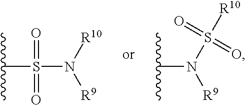

wherein R⁹ and R¹⁰ independently represents hydrogen or hydrocarbyl.

The term “sulfoxide” is art-recognized and refers to the group—S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

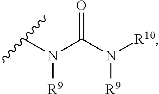

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 2001/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### Methods

The method provided herein comprise preventing or treating aneurysms (e.g., AAA, a cerebral aneurysm, or TAA) by conjointly administering a folate compound and a calcium channel blocker to a subject in need thereof. In some aspects, the methods comprise ameliorating at least one symptom or pathology of an aneurysm, comprising conjointly administering a folate compound and a calcium channel blocker to a subject in need thereof. The symptom or pathology may be, for example, increased superoxide production, increased eNOS uncoupling activity, decreased nitric oxide (NO) bioavailability, decreased tetrahydrobiopterin (H₄B) bioavailability, enlargement of blood vessels (abdominal aortas, thoracic aortas or blood vessels in the brain), increased vascular remodeling, increased elastin degradation (flattening and breakdown), increased vascular inflammation/macrophage infiltration, increased matrix metalloproteinase (MMP) activation, increased adventitial hypertrophy, or a decrease in eNOS function.

Also provided herein are methods of decreasing superoxide production, eNOS uncoupling activity, enlargement of blood vessels (abdominal aortas, thoracic aortas or blood vessels in the brain), vascular remodeling, elastin degradation (flattening and breakdown), vascular inflammation/macrophage infiltration, matrix metalloproteinase (MMP) activation, and/or adventitial hypertrophy in a subject afflicted with an aneurysm, comprising conjointly administering a folate compound and a calcium channel blocker to a subject in need thereof. In some embodiments, provided herein are methods of increasing eNOS function, and nitric oxide (NO) and tetrahydrobiopterin bioavailabilities in a subject afflicted with an aneurysm by conjointly administering a folate compound and a calcium channel blocker to a subject in need thereof.

In some embodiments, the calcium channel blocker is a dihydropyridine compound. In some embodiments, folate compound is folic acid. The calcium channel blocker and folate compound may be in same or separate compositions. The folate compound and the dihydropyridine compound may be in the same composition. The calcium channel blocker may be Nifedipine.

In some embodiments, the folate compound and the calcium channel blocker are administered in therapeutically effective amounts (e.g., an amount that is or expected to be synergistic).

The folate compound and the calcium channel blocker may be administered in a folate compound to calcium channel blocker mass ratio of about 10:1 to about 1:10, or about 100:1, about 95:1, about 90:1, about 85:1, about 80:1, about 75:1, about 70:1, about 65:1, about 60:1, about 55:1, about 50:1, about 45:1, about 40:1, about 35:1, about 30:1, about 29:1, about 28:1, about 27:1, about 26:1, about 25:1, about 24:1, about 23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 0.95:1, about 0.9:1, about 0.85:1, about 0.8:1, about 0.75:1, about 0.7:1, about 0.65:1, about 0.6:1, about 0.55:1, about 0.5:1, about 0.45:1, about 0.4:1, about 0.35:1, about 0.3:1, about 0.25:1, about 0.2:1, about 0.15:1, about 0.1:1, about 0.05:1, or about 0.01:1. The folate compound and the calcium channel blocker may be administered in a folate compound to calcium channel blocker mass ratio of about 3:1 to about 1:3.

The calcium channel blocker and folate compound may be administered in a calcium channel blocker to folate compound mass ratio of about 100:1, about 95:1, about 90:1, about 85:1, about 80:1, about 75:1, about 70:1, about 65:1, about 60:1, about 55:1, about 50:1, about 45:1, about 40:1, about 35:1, about 10:1 to about 1:10, such as about 30:1, about 29:1, about 28:1, about 27:1, about 26:1, about 25:1, about 24:1, about23:1, about 22:1, about 21:1, about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1 about 0.95:1, about 0.9:1, about 0.85:1, about 0.8:1, about 0.75:1, about 0.7:1, about 0.65:1, about 0.6:1, about 0.55:1, about 0.5:1, about 0.45:1, about 0.4:1, about 0.35:1, about 0.3:1, about 0.25:1, about 0.2:1, about 0.15:1, about 0.1:1, about 0.05:1, or about 0.01:1. The calcium channel blocker and folate compound may be administered in a calcium channel blocker to folate compound mass ratio of 1:3 or 3:1.

In some embodiments, the folate compound is administered in an amount of about 1-350 mg, about 1-700 mg, about 1-1050 mg, about 1-1400 mg, or about 1-1750 mg. The folate compound may be administered in an amount of at least or about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, or 2000 milligrams total, mg/kg body weight of the subject, or mg/kg/day.

In some embodiments, the calcium channel blocker is administered in an amount of about 1-350 mg, about 1-700 mg, about 1-1050 mg, about 1-1400 mg, about 1-1750 mg, about 1-2100 mg, or about 1-2450 mg. In some embodiments, the calcium channel blocker is administered in an amount of at least or about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, or 3000 milligrams total, mg/kg body weight of the subject, or mg/kg/day.

The folate compound and the calcium channel blocker may be administered simultaneously or sequentially. The folate compound and the calcium channel blocker may be administered conjointly. The folate compound and the calcium channel blocker may be administered in separate dosage forms.

The aneurysm may be an abdominal aortic aneurysm, cerebral aneurysm, or thoracic aortic aneurysm.

The compositions (e.g., a folate compound or a calcium channel blocker) may be administered to the subject systemically, intravenously, subcutaneously, intramuscularly, orally or locally (e.g., locally to the tumor in the subject).

In some embodiments, the folate compound and the calcium channel blocker are administered conjointly. In some embodiments, administering conjointly comprises administering the folate compound and the calcium channel blocker at different times. In some embodiments, administering conjointly comprises administering the folate compound and the calcium channel blocker concurrently. In some embodiments, administering conjointly comprises administering the folate compound before the calcium channel blocker. In some embodiments, administering conjointly comprises administering the folate compound after the calcium channel blocker.

The compositions (e.g., a folate compound or a calcium channel blocker) disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, at least ten years, at least 20 years, at least 30 years, at least 40 years, at least 50 years. The dose may be administered when needed, sporadically, or at regular intervals. For example, the dose may be administered monthly, weekly, biweekly, triweekly, once a day, twice a day, or three times a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, a dose of the composition is administered at least three times a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, at least five years, at least 10 years, at least 20 years, at least 30 years, at least 40 years, at least 50 years.

Toxicity and therapeutic efficacy of the combination of the one or more folic acid compounds and one or more calcium channel blocker compounds according to the instant invention and compositions thereof can be determined using cell cultures and/or experimental animals and pharmaceutical procedures in the art. For example, one may determine the lethal dose, LC50 (the dose expressed as concentration x exposure time that is lethal to 50% of the population) or the LD50 (the dose lethal to 50% of the population), and the ED50 (the dose therapeutically effective in 50% of the population) by methods in the art. The dose ratio between toxic and therapeutic effects is the therapeutic index and it can be expressed as the ratio LD50/ED50. Folic acid compounds and calcium channel blocker compounds which exhibit large therapeutic indices are preferred. While combinations of folic acid compounds and calcium channel blocker compounds that result in toxic side-effects may be used, care, may be taken to design a delivery system that targets such compounds to the site of treatment to minimize potential damage to uninfected cells and, thereby, reduce side-effects.

The data obtained from cell culture assays and animal studies can be used in formulating a range of dosages for use in humans. Preferred dosages provide a range of circulating concentrations that include the ED50 with tolerated, little or no toxicity. The dosage may vary depending upon the dosage form employed and the route of administration utilized. Therapeutically effective amounts and dosages of one or more folic acid compounds and one or more, for example, Nifedipine compounds can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

Actual dosage levels of the active ingredients in the pharmaceutical compositions or agents to be administered may be varied so as to obtain an amount of the active ingredient (e.g., an agent described herein) which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could prescribe and/or administer doses of the compounds employed in the pharmaceutical composition at levels lower than that required in order to achieve the desired therapeutic effect and gradually increase the dosage until the desired effect is achieved.

### Kits

In some embodiments, the present invention provides kits comprising one or more folic acid compounds and one or more calcium channel blockers, optionally in a composition, packaged together with one or more reagents or drug delivery devices for preventing, inhibiting, reducing, or treating aneurysms in a subject. In some embodiments, the kits comprise one or more folic acid compounds and one or more calcium channel blocker compounds, optionally in one or more unit dosage forms, packaged together as a pack and/or in drug delivery device, e.g., a pre-filled syringe. The one or more folic acid compounds and/or the one or more calcium channel blocker compounds may be provided in the form of a pharmaceutical composition. In some embodiments, the one or more folic acid compounds and/or the one or more calcium channel blocker compounds are provided in the form of an oral formulation, e.g., a tablet.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### EXAMPLES

The invention now being generally described, it will be more readily understood by reference to the following examples which are included merely for purposes of illustration of certain aspects and embodiments of the present invention, and are not intended to limit the invention.

Aortic aneurysms are prevalent and severe vascular diseases, with high mortality resulting from patients dying of unpredictable sudden rupture of the aneurysms. No treatment options have been generally available except for surgical correction of large aneurysms, which presents considerable risk. The data demonstrate that orally administrated comminatory therapy of FA and Nifedipine can serve as a robust, first-in-class, and effective treatment regime for aortic and other aneurysms.

### Example 1: Combination of Folic Acid with Nifedipine Substantially and Completely Further Attenuates Formation of Aortic Aneurysm in a Dose-dependent Manner

Applicant has shown that folic acid (FA) is highly effective in alleviating aortic aneurysm formation (both AAA and TAA) in Angiotensin II (Ang II) infused apolipoprotein E (apoE) null mice, Ang II infused hyperphenylalaninemia (hph-1) mice and Fbnl Marfan Syndrome mice, via recoupling of eNOS to attenuate superoxide production while restoring nitric oxide (NO) bioavailability, resulting in abrogated vascular remodeling. Nonetheless, FA alone is not sufficient to completely attenuate development of aortic aneurysms. Here, the therapeutic effects on aortic aneurysm of combining FA with Nifedipine are examined. Oral administration with FA (15 mg/kg/day) significantly reduced incidence of AAA from 85.71% to 18.75% in Ang II-infused apoE null mice, while combination of FA with various doses of Nifedipine (1.5, 5.0 or 20 mg/kg/day) substantially and completely further reduced the incidence to 12.5%, 11.76% and 0.00% respectively in a dose-dependent manner. The combinatory therapy substantially and completely further alleviated enlargement of abdominal aortas defined by ultrasound also in a dose-dependent manner, with combination of FA with 20 mg/kg/day Nifedipine attenuating sizes of abdominal aortas by 100% to control levels. Vascular remodeling featured by elastin degradation and adventitial hypertrophy was abolished by combinatory therapy dose-dependently. Combination of FA with Nifedipine substantially and completely further diminished electron spin resonance (ESR) determined total superoxide production and eNOS uncoupling activity in a dose- dependent manner, which was accompanied by dose-dependent further improvement in aortic NO and H₄B bioavailabilities.

Oxidative stress has been shown to play an important role in the formation of aortic aneurysms including AAA and thoracic aortic aneurysm (TAA). Uncoupled endothelial nitric oxide synthase (eNOS) plays a critical role in AAA formation via sustaining oxidative stress to induce matrix metalloproteinase (MMP) activation and matrix degradation. It was first demonstrated that eNOS uncoupling mediates AAA formation in a novel model of AAA, namely angiotensin II (Ang II) infused hph-1 mice in which 79% of the mice developed AAA within 2 weeks of Ang II infusion, with 14% died of rupture aneurysm. The Ang II infused hph-1 mice prove to be the most robust AAA model to date. Restoration of dihydrofolate reductase (DHFR) function with folic acid (FA) to recouple eNOS markedly attenuated AAA formation in these animals. Moreover, it has been further demonstrated that eNOS uncoupling plays a novel role in the development of AAA in Ang II-infused apoE null mice, a well-established, classical model of AAA, while oral FA administration also effectively restored DHFR function to recouple eNOS, resulting in abrogated aneurysm formation. Indeed, knockout of DHFR in mice facilitating uncoupling of eNOS leads to exaggerated formation of AAA. It has further been shown that activation of NADPH oxidase (NOX) isoforms lies upstream of uncoupled eNOS to drive AAA formation. Furthermore, eNOS uncoupling also plays a critical role in driving TAA and AAA formation in Fbnl Marfan Syndrome mice, a classical model for TAA, which can also be targeted by FA to alleviate formation of both TAA and AAA (Huang et al., Redox Biology, (2021)). Therefore, these data further confirm a critical causal role of eNOS uncoupling in AAA and TAA formation, while also establishing a universal efficacy of FA in treating aortic aneurysms in different animal models, via restoration of eNOS coupling activity to attenuate oxidative stress and consequent vascular remodeling. Likewise, oxidative stress-driven vascular remodeling is a common pathway shared between AAA/TAA and cerebral aneurysm, so that FA would be equivalently effective in treating cerebral aneurysm. Nonetheless, FA alone is not sufficient to fully alleviate formation of aortic aneurysms.

Hypertension is a risk factor for aortic aneurysms, and AAA and TAA patients often have co-existing hypertension. It is also a major risk factor for cerebral aneurysm. Previous studies have investigated effects on AAA formation of both low and high doses of the anti-hypertensive drug Nifedipine, which is a calcium channel blocker. Low doses of Nifedipine have no effect on blood pressure, however, both low and high doses of Nifedipine can attenuate AAA formation, via restoration of DHFR and inhibition of NOX to attenuate eNOS uncoupling. The high dose of Nifedipine can also reduce blood pressure at the same time, hence particularly valuable to treat aortic aneurysm patients with co-existing hypertension. Nonetheless, neither FA nor Nifedipine alone is sufficient to fully alleviate development of AAA. The data shown herein examines whether combination of FA with various doses of Nifedipine has augmented efficacies in treating AAA. Ang II-infused apoE null mice are subjected to oral administration of FA (15 mg/kg/day) and FA in combination with increasing doses of Nifedipine at 1.5, 5.0 or 20.0 mg/kg/day. Remarkably, the data indicate that combination of FA with various doses of Nifedipine substantially and completely further alleviated AAA formation in a dose-dependent manner. The combinatory therapies were robustly more effective in restoring eNOS coupling activity to improve NO bioavailability and attenuate oxidative stress, resulting in abrogated vascular remodeling characterized by elastin degradation and adventitial hypertrophy, with combination of FA with Nifedipine substantially and completely further inhibiting incidence of AAA in a dose-dependent manner from 18.75% in FA group (reduced from 85.71% in Ang II infused apoE null mice) to 12.50%, 11.76% and 0.00% respectively for groups of FA plus 1.5, 5.0 or 20 mg/kg/day Nifedipine. The enlargement of abdominal aortas defined by echocardiography was substantially and completely further attenuated by combining FA with Nifedipine in a dose-dependent manner, with the FA combined with high dose of Nifedipine at 20 mg/kg/day alleviating expansion of abdominal aortic areas by 100% to control levels.

### Combination of FA with Nifedipine Substantially and Completely Further Attenuated Incidence of AAA in a Dose-Dependent Manner

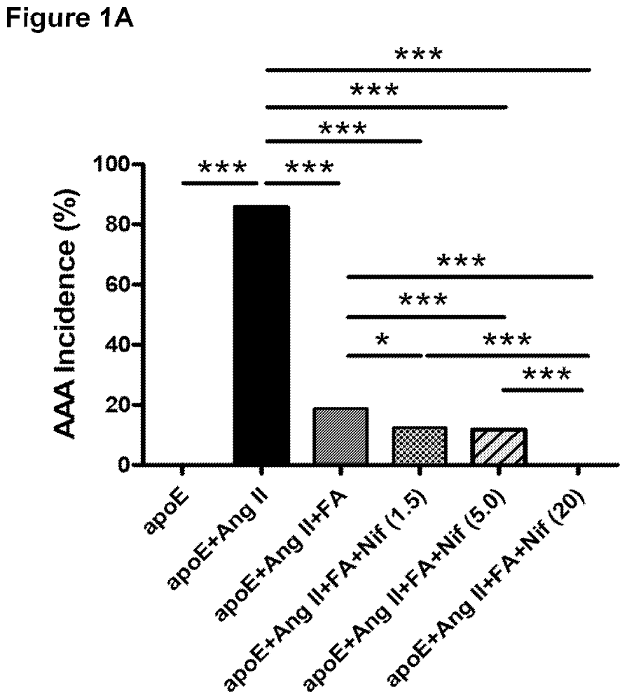

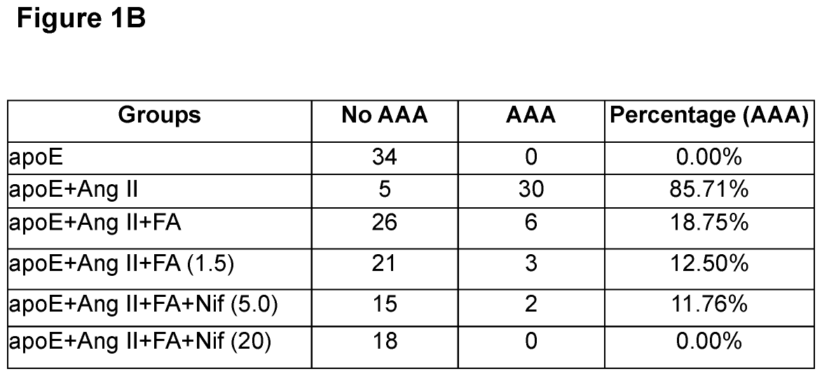

The incidence of AAA was examined in Ang II infused apoE null mice subjected to oral administration of FA alone, or combinatory therapy of FA with various doses of Nifedipine (1.5, 5 or 20 mg/kg/day). As shown in FIG. 1A-1B, after 4 weeks of Ang II infusion, 85.71% (30 out of 35) of the Ang II infused apoE null mice developed AAA. Oral FA administration significantly reduced incidence of AAA to 18.75% (p<0.001, 6 out of 32). Compared to FA alone group, combination of FA with various doses of Nifedipine (1.5, 5 or 20 mg/kg/day) substantially and completely further reduced incidence of AAA to 12.50% (p<0.05, 3 out of 24), 11.76% (p<0.001, 2 out of 17) and 0.00% (p<0.001, 0 out of 18) respectively in a dose-dependent manner, indicating that combinatory therapy of FA and Nifedipine is robustly more effective in treating aortic aneurysms.

### Combination of FA with Nifedipine Substantially and Completely Further Attenuated Enlargement of Abdominal Aortas in a Dose-Dependent Manner

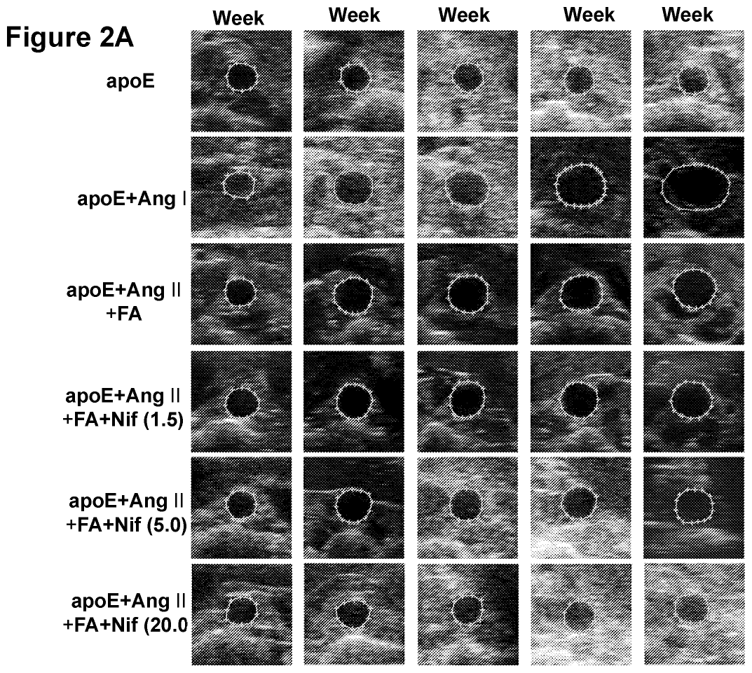

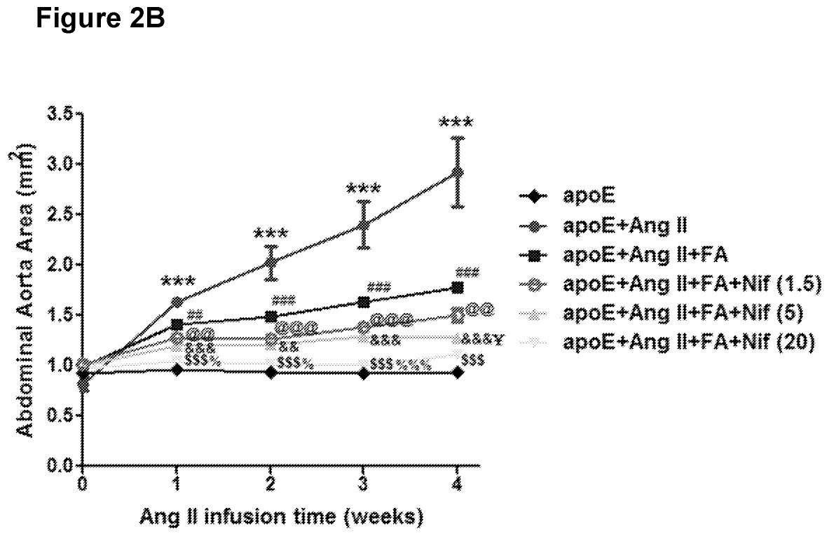

To examine enlargement of abdominal aortas in Ang II infused apoE null mice that were treated with FA alone, or FA in combination with 1.5, 5 or 20 mg/kg/day Nifedipine, sizes of abdominal aortas were monitored weekly using ultrasound. FIG. 2A shows representative ultrasound images of abdominal aortas from all experimental groups across the 4 weeks, while FIG. 2B shows grouped data. Of note, abdominal aortas of Ang II infused apoE null mice were markedly enlarged comparing to untreated apoE null mice, which was significantly attenuated by oral administration of FA (FIGS. 2A-2B, ##p<0.01 or ###p<0.001 vs. apoE+Ang II of the same week). Remarkably, combination of FA with 1.5, 5 or 20 mg/kg/day Nifedipine substantially or completely further attenuated expansion of abdominal aortas in a dose-dependent manner, with the FA plus 20 mg/kg/day Nifedipine group showing reduction in aortic enlargement by 100% to control levels (FIGS. 2A-2B, @@p<0.01 or @@@p<0.001 for 1.5 mg/kg/day Nifedipine group; &&p<0.01 or &&&p<0.001 for 5.0 mg/kg/day Nifedipine group; $$$p<0.001 for 20 mg/kg/day Nifedipine group). For some time points (should still keep this? Since differences not for all time points; also we want to draw attention to the fact even 1.5 was more than sufficient to be more effective; or remove this sentence including the latter half?), Nifedipine 5.0 or 20 mg/kg/day group was significantly more effective than Nifedipine 1.5 mg/kg/day group (¥p<0.05; %p<0.05 or %%% p<0.001)

### Combination of FA and Nifedipine subStantially and Completely Further Attenuated Vascular Remodeling in a Dose-Dependent Manner

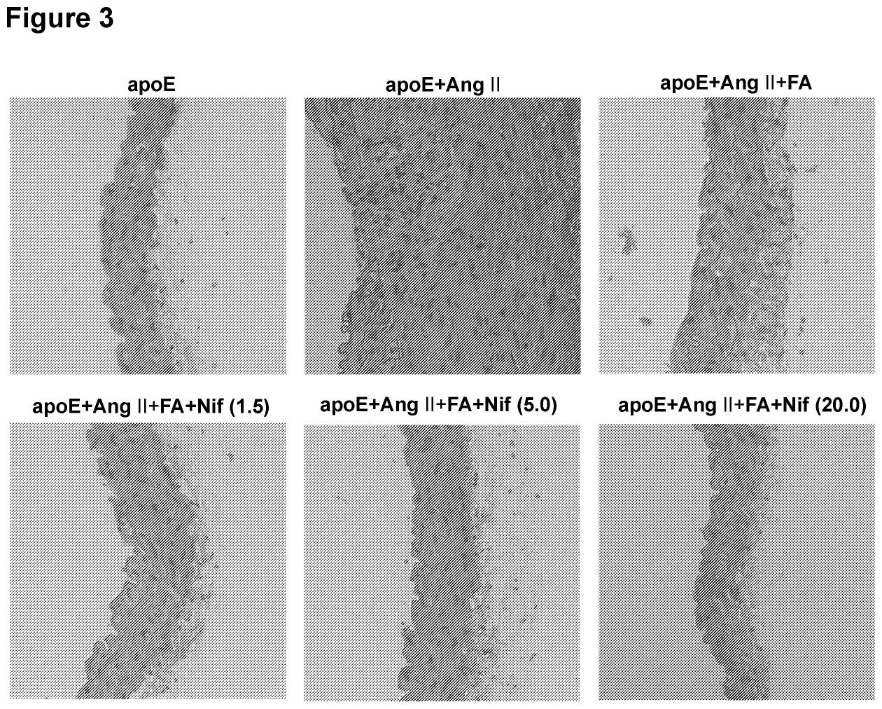

Formation of aortic aneurysms is accompanied by extensive vascular remodeling featured by matrix degradation to allow expansion of aortas. To examine the extent of the vascular remodeling that occurred during AAA in the presence of various treatments, freshly isolated aortas were embedded in paraffin, sectioned and stained with hematoxylin-eosin (H&E). As shown in FIG. 3, oral administration of FA markedly attenuated vascular remodeling featured by elastic degradation and adventitial hypertrophy in Ang II infused apoE null mice. Combination of FA with 1.5, 5 or 20 mg/kg/day Nifedipine substantially and completely further attenuated vascular remodeling in a dose-dependent manner.

### Combination of FA with Nifedipine Substantially and Completely Further Attenuate Elastin Degradation in a Dose-Dependent Manner

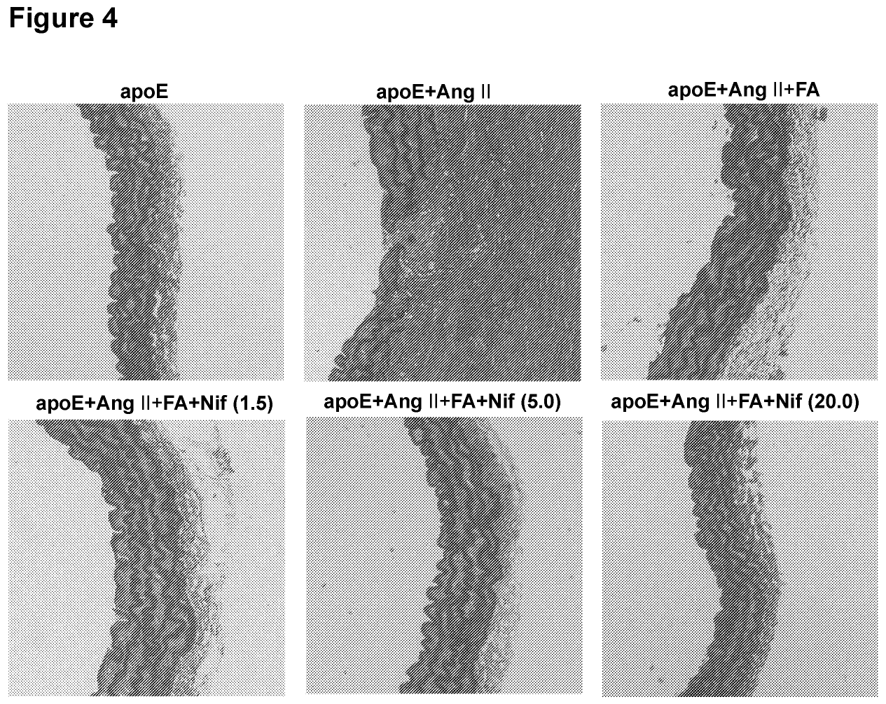

To assess medial elastin degradation, VVG staining was performed using aortic sections from all experimental groups. As shown in FIG. 4, elastin degradation featured by flattening and breakdown was obvious in Ang II infused apoE null mice, which was significantly attenuated by oral administration of FA. Importantly, combination of FA with various doses of Nifedipine (1.5, 5 or 20 mg/kg/day) substantially and completely further attenuated elastin degradation in a dose-dependent manner.

### Combination of FA with Nifedipine Substantially and Completely Further Attenuated Total Superoxide Production and eNOS Uncoupling Activity in a Dose-Dependent Manner

eNOS uncoupling takes on a mediator role of in AAA formation in both novel and classical models of Ang II infused hph-1 and apoE null mice, as well as in TAA formation in a classical model of Fbnl Marfan Syndrome mice. Therefore, changes in aortic total superoxide production and eNOS uncoupling activity from all treatment groups is examined herein. Freshly prepared aortic lysates were subjected to electron spin resonance (ESR) determination of superoxide production with and without L-NAME, an inhibitor of NOS. If eNOS is functional and coupled, its inhibition by L-NAME will increase the measured superoxide, as eNOS is producing NO to scavenge superoxide. However, if eNOS is dysfunctional, uncoupled and producing superoxide, its inhibition with L-NAME will lead to a decrease in measured superoxide.

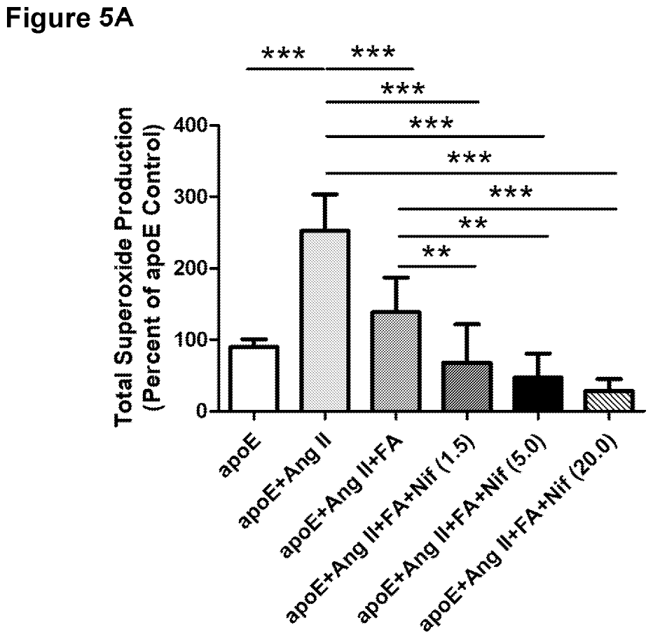

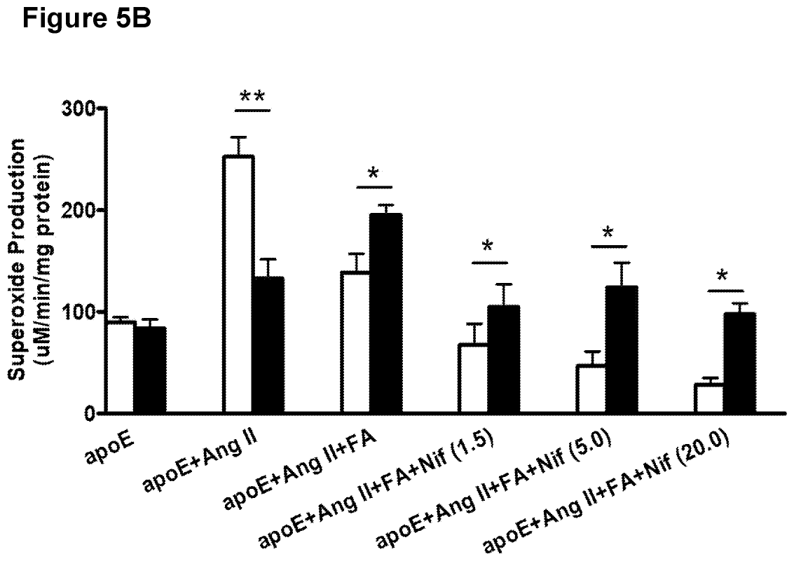

As is shown in FIG. 5A, there was a significant increase in total superoxide production in aortas isolated from Ang II infused apoE null mice, which was markedly abrogated by oral FA administration. This represents the molecular mechanism underlying protective effects in FA on AAA formation. Intriguingly, combination of FA with various doses of Nifedipine (1.5, 5 or 20 mg/kg/day) substantially and completely further attenuated total superoxide production in a dose-dependent manner.

As shown in FIG. 5B, there was a marked increase in eNOS uncoupling activity in Ang II infused apoE null mice. At baseline, eNOS is minimally uncoupled in apoE null mice that is well-compensated. Oral administration with FA reversed eNOS uncoupling activity in Ang II infused apoE null mice (FIG. 5B). Combination of FA with 1.5, 5 or 20 mg/kg/day Nifedipine also completely attenuated eNOS uncoupling activity while abrogating total superoxide production more effectively in a dose-dependent manner (FIGS. 5A-5B). These data indicate that recoupling of eNOS and further reduction in total superoxide production underlie improved efficacies in attenuating AAA formation by combinatory therapy of FA and Nifedipine.

### Combination of FA with Nifedipine Substantially Further Improved NO Bioavailability in a Dose-Dependent Manner

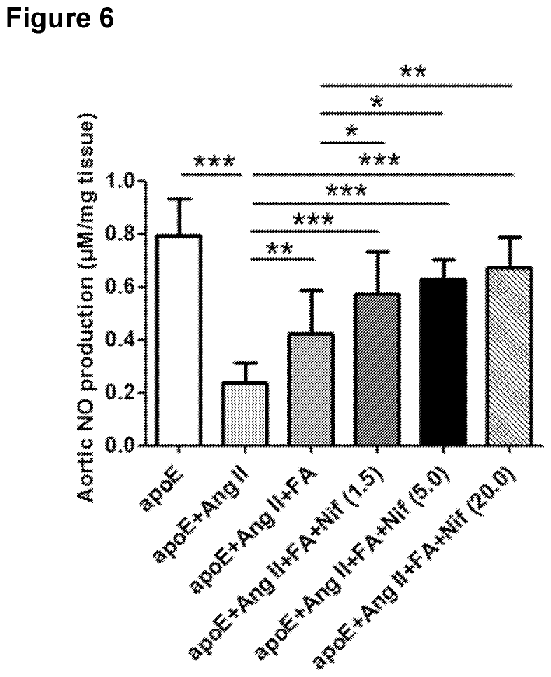

Since FA plus Nifedipine recoupled eNOS to diminish superoxide production as described above, aortic NO bioavailability from all experimental groups using electron spin resonance (ESR) was examined next. The data indicate that there was a significant decrease in NO bioavailability in aortas isolated from Ang II infused apoE null mice, which was significantly restored by oral FA administration (FIG. 6). Intriguingly, combination of FA with various doses of Nifedipine (1.5, 5 or 20 mg/kg/day) substantially further improved aortic NO bioavailability in a dose-dependent manner, going along with restoration of eNOS function to mediate protection against formation of AAA.

### Combination of FA with Nifedipine Substantially Further Improved Aortic H₄B Bioavailability in a Dose-Dependent Manner

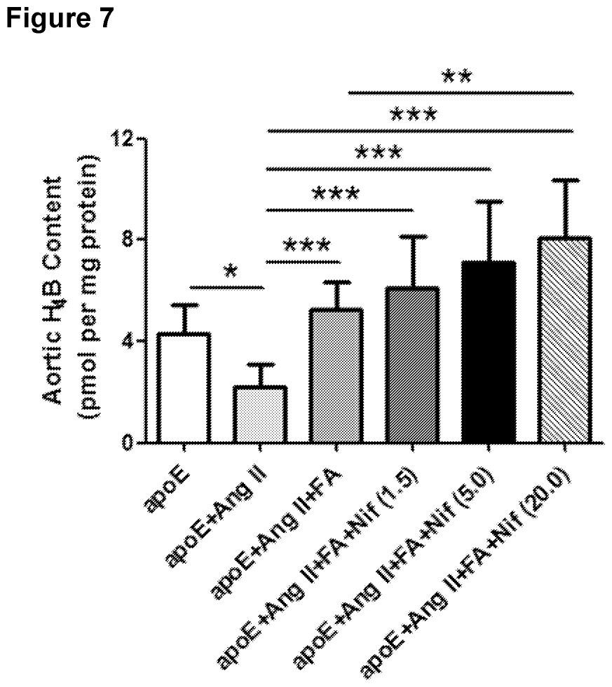

As an essential cofactor of eNOS, H₄B deficiency is indicative of eNOS uncoupling. Herein, it was determined that aortic H₄B bioavailability from all experimental groups using HPLC. As shown in FIG. 7, Ang II induced H₄B deficiency in apoE null mice was significantly abrogated by oral FA administration, while combination of FA with various doses of Nifedipine (1.5, 5 or 20 mg/kg/day) substantially further improved aortic H₄B bioavailability in a dose-dependent manner. Taken together with data describe above, these findings strongly indicate that combination of FA and Nifedipine over several doses proves to be an effective therapeutic regime for AAA, via attenuation of eNOS uncoupling activity to abrogate oxidative stress and consequent vascular remodeling.

### Discussion

The data shows herein demonstrates for the first time that combinatory therapy of FA and Nifedipine, attenuates formation of aortic aneurysm in a model of Ang II-infused apoE null mice, with the addition of Nifedipine showing a dose-dependent effect in augmenting the efficacy of FA in treating aortic aneurysm. AAA incidence determined by ultrasound definition of aortic expansion and post-mortem inspection was synergistically, as well as substantially and completely, further abrogated by combination of FA with Nifedipine in a dose-dependent manner. Combinatory therapy of FA and Nifedipine substantially and completely further alleviated vascular remodeling featured by elastin degradation and adventitial hypertrophy in a dose-dependent manner. These protective effects on AAA incidence and related pathophysiological changes are attributed to augmented effects of combinatory therapy on restoration of eNOS function and abrogation of oxidative stress. Combination of FA with various doses of Nifedipine substantially and completely further attenuated superoxide production in a dose-dependent manner while completely recoupling eNOS to restore NO bioavailability also in a dose-dependent manner. The aortic bioavailability of eNOS cofactor H₄B was indeed further improved by the combinatory therapy, going along with the outcome of eNOS recoupling. These data strongly demonstrate that combinatory therapy of FA and Nifedipine can serve as a novel oral medication in the treatment of aneurysms, especially considering the common oxidative stress pathway shared between AAA/TAA and cerebral aneurysm.

Oxidative stress plays a role in the pathogenesis of AAA. Specifically, as shown by the data herein, uncoupled eNOS mediates formation of AAA by driving oxidative stress and consequent vascular remodeling. It is demonstrated that, in Ang II infused hph-1 mice, a newly established, robust model of AAA, uncoupling of eNOS mediates AAA formation that can be attenuated by FA restoration of endothelial dihydrofolate reductase (DHFR) function/H₄B bioavailability to recouple eNOS. It is also shown herein, in a classical AAA model of Ang II-infused apoE null mice, eNOS uncoupling mediates sustained oxidative stress and AAA formation that can be alleviated by oral FA administration. In both Ang II-infused hph-1 and apoE null mice, oral FA administration is effective in reducing incidence of AAA, preventing enlargement of abdominal aorta, and alleviating maladaptive vascular remodeling. This is mediated by FA recoupling of eNOS to attenuate superoxide production while restoring NO bioavailability, resulting in abrogated oxidative stress by shutting down the enzymatic system of uncoupled eNOS . In addition, it has been recently shown that eNOS uncoupling plays a similar causal role in TAA formation in a classical model of Fbnl Marfan Syndrome mice, in which recoupling of eNOS with FA diet also attenuated formation of TAA and AAA. Data from the present study further confirm these findings to establish a critical role of eNOS uncoupling in formation of aortic aneurysms including AAA and TAA, targeting of which by FA proves to be a highly effective treatment option. Nonetheless, FA alone cannot completely attenuate aneurysm formation. It is therefore proposed that combinatory therapy of FA with another class of anti-hypertensive drug of Nifedipine, shown in previous studies to be effective in alleviating AAA formation as well, will have synergistic and augmented effects in treating AAA. The role of eNOS uncoupling/oxidative stress in mediating aneurysm formation is a common mechanism for both AAA and TAA. Therefore, the combination treatment disclosed herein can be used to prevent or treat TAA. The combination treatments disclosed herein would also be synergistic when administered to an individual with TAA. Likewise, it would also be synergistically effective in treatment cerebral aneurysm.

Therefore, these data show Ang II-infused apoE null mice treated with combinatory therapy of FA and various doses of Nifedipine to examine potential synergistic and augmented effects of the treatment on attenuating aneurysm formation. FA combined with 1.5, 5 or 20 mg/kg/day Nifedipine substantially and completely further reduced incidence of AAA to 12.50%, 11.76% and 0.00% respectively in a dose-dependent manner, which represent substantially improved efficacies comparing to FA alone (18.75%). The combinatory therapy further abrogated expansion of abdominal aortas in a dose-dependent manner compared to the FA alone group, with FA plus 20 mg/kg/day of Nifedipine attenuating sizes of the abdominal aortas by 100% to control levels, again confirming a substantial further improvement in the efficacy of treating AAA. Moreover, vascular remodeling featured by elastin degradation and adventitial hypertrophy was also substantially and completely further alleviated in FA plus Nifedipine groups in a dose-dependent manner.

Combinatory therapy of FA and various doses of Nifedipine substantially and completely further improved efficacy of FA in attenuating total superoxide production and eNOS uncoupling activity in a dose-dependent manner. Though the treatment with FA more directly targets the coupling state of eNOS, Nifedipine targets a seemingly unrelated pathway of calcium channel in the cell. Nifedipine has an inhibitory effect on NADPH oxidase that is upstream of uncoupled eNOS, besides its upregulating effects on DHFR. This differential mechanism might explain the synergistic effects between FA and Nifedipine in attenuating aortic aneurysm formation via enhanced efficacies in restoration of eNOS function/NO bioavailability to abrogate oxidative stress and consequent vascular remodeling. Indeed, H₄B deficiency as an indicator of eNOS uncoupling activity, was also markedly further alleviated by combination of FA with various doses of Nifedipine in a dose-dependent manner.

The data disclosed herein strongly demonstrate that combination of FA with various doses of Nifedipine substantially and completely further attenuates formation of aortic aneurysms and related pathophysiological and molecular changes in a dose-dependent manner. Oral administration of FA and Nifedipine thus proves to be an effective oral medicine for the treatment of aneurysms including AAA/TAA and cerebral aneurysm which are driven by the common pathway of eNOS uncoupling/oxidative stress, with composition containing high dose of Nifedipine particularly suitable for treatment of aneurysm patients with co-existing hypertension.

### Materials and Methods for Example 5

### Animals

The use of animals and experimental procedures were approved by the Institutional Animal Care and Usage Committee (IACUC) at the University of California Los Angeles (UCLA). Breeders of apoE null mice were purchased from Jackson Labs (Bar Harbor, ME, Strain B6.129P2-ApoEtmlUnc/J), and bred in house. Animals were kept in ventilated cages, with free access to water and standard chow under the standard care of Division of Laboratory Animal Medicine (DLAM) staff. Male apoE null mice of 6-8 months old were used for experimentation.

Ang II infusion by osmotic minipump

The animals receiving Ang II infusion were anesthetized in an isoflurane chamber with 5% isoflurane, and then moved to a nose cone with sustained supply of 1.5-2% isoflurane to maintain the anesthetic state. The back area between the shoulder blades was cleaned of hair and disinfected. Then a small incision was made at the cleaned site; the osmotic minipump (Alzet, model 2004) containing Ang II (1000 ng/kg/min, Sigma-Millipore, St. Louis, MO, USA) was subcutaneously implanted into the mice. The surgical wounds were closed with surgical staples, and then animals were allowed to recover in a heated cage.

### Oral Administration of Folic Acid (FA) and Nifedipine

For animal groups orally treated with folic acid (FA) or FA in combination with various doses of Nifedipine, standard chow was replaced with customized food tablets containing FA (15 mg/ kg/day) alone, or FA in combination with Nifedipine (1.5, 5 or 20 mg/kg/day) two days prior to implantation of osmotic minipumps for Ang II infusion, and throughout the study period of 4 weeks of Ang II infusion.

### Ultrasound Detection of Abdominal Aortic Size

The enlargement of abdominal aortas in various experimental groups was monitored using ultrasound. Animals were anesthetized with isoflurane and placed on a temperature-controlled table. Isoflurane levels were adjusted throughout the experiment to maintain heart rate between 400 and 500 bpm while keeping the animal sufficiently anesthetized. Hair was removed from the abdomen using a hair removal cream, and preheated ultrasound transmission gel was applied onto the abdomen area. An ultrasound probe was placed on the gel to visualize aorta transversely (Vevo 2100, MS400, 30 MHz. FUJIFILM VisualSonics, Inc., Toronto, Ontario, Canada). The aorta was identified using Doppler measurement for the presence of pulsatile flow. Consistent localization of abdominal aortas for image acquisition was insured by visualizing the aorta immediately superior to the branch of the left renal artery in all of the animals.

### Anatomical Inspection of Abdominal Aortas and Histological Analyses

At the end of the study period of 4 weeks, animals were euthanized with CO2. The aortas were rapidly removed from the body, rinsed with ice cold Krebs/HEPES buffer, and cleaned of connective tissue and fat. The incidence of AAA was determined by ultrasound assessment of abdominal aortic expansion as described above, and by direct inspection of the abdominal aortas post-mortem. For histological analyses, small sections (5 mm) of the abdominal aortas of the suprarenal region were removed and fixed in 4% paraformaldehyde overnight, followed by incubation for 24 hours in 10% sucrose, and then embedded in paraffin. In the case of AAA, a center section of the AAA was used for these analyses. Sections were sliced at 5μm at UCLA Pathology Core, and subjected to hematoxylin-eosin (H&E) staining following standard protocol.

### Verhoeff-Van Gieson (VVG) Staining

Paraffin embedded aortic sections were deparaffinized by sequential washes in xylene (2x), descending ethanol from 100%, 90%, 75% to 50%, and distilled water. Sections were stained in Verhoeff s solution for 70 min, followed by differentiation in 2% ferric chloride for 90 seconds. Sections were incubated with 5% sodium thiosulfate for 60 seconds, followed by counterstaining with Van Gieson's solution. Sections were then subjected to dehydration with 95% and 100% alcohol, and finally washed in xylene. After drying, sections were mounted with Permount mounting media (SP15-100, Thermo-Fisher Scientific, Pittsburgh, PA, USA), and images captured by a Nikon TE2000-U fluorescent microscope.

Electron Spin Resonance Determination of Total Aortic Superoxide Production and eNOS Uncoupling Activity

Aortic superoxide production was determined by electron spin resonance (ESR). In brief, freshly isolated aortas were homogenized in lysis buffer containing 1:100 protease inhibitor cocktail (MilliporeSigma, P8340), centrifuged at 12,000 rpm for 15 min, and protein supernatant collected. After determination of protein concentration using a protein assay kit (Bio-Rad), five μg of the protein was loaded into ice-cold and nitrogen bubbled modified Krebs/HEPES buffer (KHB, in mmol/L: NaCl 99; KCl 4.7; MgSO41.2; KH₂PO4 1.0; CaCl2 1.9; NaHCO3 25; glucose 11.1, NaHEPES 20) containing diethyldithiocarbamic acid (5 mmol/L), deferoxamine (25 mmol/L), and the superoxide specific spin trap methoxycarbonyl-2,2,5,5-tetramethylpyrrolidine (CMH, 500 μmol/L, Axxora, San Diego, CA, USA). The mixture was loaded into a glass capillary (Kimble, 71900-50, Dover, OH, USA), and assayed using electron spin resonance (ESR) spectrophotometer (eScan, Bruker, Billerica, MA, USA) for total superoxide production by taking the difference in the presence or absence of PEG-SOD (polyethylene glycol-superoxide dismutase; 100 U/mL, MilliporeSigma, St. Louis, MO, USA). To determine eNOS uncoupling activity, measurements were made with the addition of L-NAME (N (ω)-nitro-L-arginine methylester; 10 μmol/L, 80587, Cayman Chemical, Ann Arbor, MI). A reduction in superoxide production with L-NAME indicates that eNOS is uncoupled producing superoxide, while an increase in superoxide production with L-NAME indicates that eNOS is coupled producing NO. The ESR settings used were: Center field, 3480; Sweep width, 9G; microwave frequency, 9.78 GHz; microwave power, 21.02 mW; modulation amplitude, 2.47 G; 512 points of resolution; receiver gain,1000.

### Electron Spin Resonance Determination of Aortic Nitric Oxide Production

Aortic nitric oxide (NO) bioavailability was determined by electron spin resonance (ESR). In brief, freshly isolated aortic rings were incubated with freshly prepared NO specific spin trap Fe²⁺(DETC)₂ (0.5 mmol/L) colloid in modified Krebs/HEPES buffer at 37° C. for 60 min, in the presence of calcium ionophore A23187 (10 mmol/L). After incubation, the aortic pieces were snap frozen in liquid nitrogen and loaded into a finger Dewar for analysis with ESR spectrophotometer (eScan, Bruker, Billerica, MA, USA). The instrument settings used were as the followings: Center field, 3440; Sweep width, 100 G; microwave frequency, 9.796 GHz; microwave power 13.26 mW; modulation amplitude, 9.82 G; 512 points of resolution; and receiver gain 356.

### HPLC Determination of Aortic H₄B Bioavailability

For determination of aortic H₄B levels freshly isolated aortas were lysed in H₄B lysis buffer (0.1 mol/L phosphoric acid, 1 mmol/L EDTA, 10 mmol/L DLDithiothreitol), centrifuged at 12,000 rpm for 10 min, and then supernatants subjected to differential oxidation in acidic (0.2 mol/L trichloroacetic acid with 2.5% I₂ and 10% KI) and alkalytic (0.1 mol/L NaOH with 0.9% I₂ and 1.5% KI) solutions. After centrifugation, 10 μl of the supernatant was injected into a HPLC system equipped with a fluorescent detector (Schimadzu America Inc, Carlsbad, CA, USA). Excitation and emission wavelengths of 350 nm and 450 nm were used to measure H₄B levels.

### Statistical Analyses

All grouped data are presented as Mean±SEM. Statistical analyses were carried out with the Prism software. Comparisons between multiple groups were done using one-way ANOVA followed by the Newman-Keuls post-hoc test. Statistical significance was set at p<0.05. Comparisons of the AAA incidence rates were done using a contingency table, also with a statistical threshold of 0.05.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A kit comprising a folate compound and a calcium channel blocker.

2. The kit of claim 1, wherein the folate compound is represented by formula I 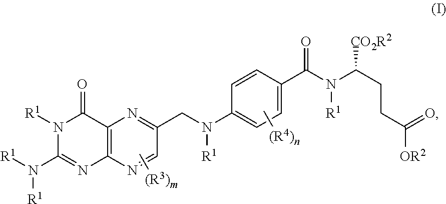
or a pharmaceutically acceptable salt thereof, wherein:
each R¹ independently is hydrogen, acyl, ester, amide, alkyl, alkenyl, alkynyl, carbocyclyl, heterocyclyl, aryl, or heteroaryl;
each R² independently is hydrogen, alkyl, alkenyl, alkynyl, carbocyclyl, heterocyclyl, aryl, or heteroaryl;
each R³ and R⁴ independently is halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl;
m is an integer selected from 0-3; and
n is an integer selected from 0-4.

3. The kit of claim 2, wherein each R¹ independently is hydrogen, acyl, ester, amide, or alkyl.

4. The kit of claim 2, wherein each R¹ is hydrogen.

5. The kit of any one of claims 2-4, wherein each R² independently is hydrogen or alkyl.

6. The kit of claim 5, wherein each R² is hydrogen.

7. The kit of any one of claims 2-6, wherein m is 0.

8. The kit of any one of claims 2-7, wherein n is 0.

9. The kit of claim 2, wherein the folate compound is 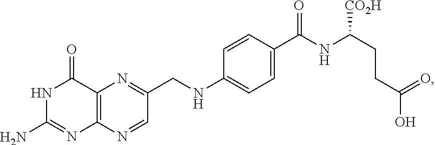 [SMILES: Nc1nc2ncc(CNc3ccc(C(=O)N[C@@H](CCC(=O)O)C(=O)O)cc3)nc2c(=O)[nH]1]
or a pharmaceutically acceptable salt thereof.

10. The kit of claim 1, wherein the calcium channel blocker is a dihydropyridine compound.

11. The kit of claim 10, wherein the dihydropyridine compound is represented by formula II: 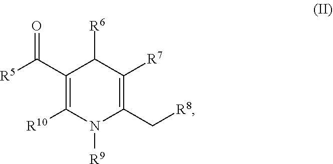
or a pharmaceutically acceptable salt thereof, wherein:
R⁵, R⁶, R⁷, R⁸, and R¹⁰ each independently is hydrogen, halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl; and
R⁹ is hydrogen, acyl, ester, amide, alkyl, alkenyl, alkynyl, carbocyclyl, heterocyclyl, aryl, or heteroaryl.

12. The kit of claim 11, wherein R⁶ is alkyl, alkenyl, alkynyl, carbocyclyl, heterocyclyl, aryl, or heteroaryl.

13. The kit of claim 12, wherein R⁶ is alkyl or aryl.

14. The kit of claim 13, wherein R⁶ is methyl or substituted or unsubstituted phenyl.

15. The kit of claim 14, wherein R⁶ is phenyl optionally substituted with halogen, haloalkyl, alkyl, or nitro.

16. The kit of claim 11, wherein the dihydropyridine compound is represented by formula II-a, II-b, II-c, or II-d:

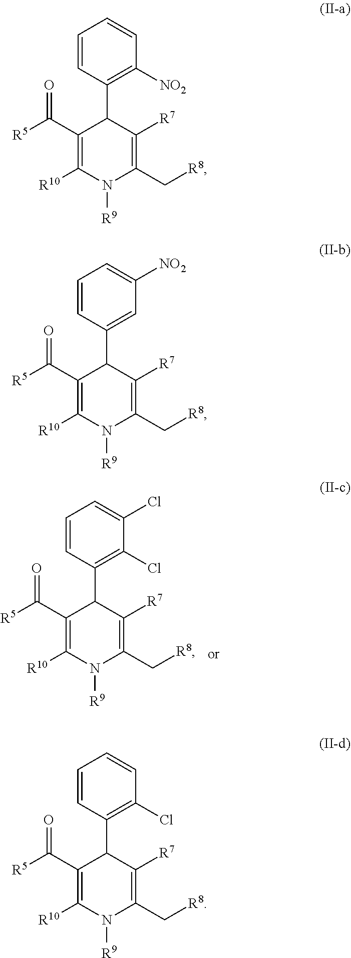

17. The kit of any one of claims 11-16, wherein R⁹ is hydrogen, alkyl, alkenyl, alkynyl, carbocyclyl, heterocyclyl, aryl, or heteroaryl.

18. The kit of claim 17, wherein R⁹ is hydrogen.

19. The kit of claim 17, wherein R⁹ is alkyl optionally substituted with halogen, amino, hydroxyl, alkoxy, cyano, nitro, acyl, ester, amide, alkylthio, carbocyclyl, heterocyclyl, aryl, or heteroaryl.

20. The kit of claim 19, wherein R⁹ is

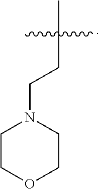 [SMILES: *CCN1CCOCC1]

21. The kit of any one of claims 11-20, wherein R¹⁰ is cyano, amino, alkyl, alkenyl, alkynyl, carbocyclyl, heterocyclyl, aryl, or heteroaryl.

22. The kit of claim 21, wherein R¹⁰ is cyano, amino, or alkyl.

23. The kit of claim 22, wherein R¹⁰ is methyl.

24. The kit of claim 11, wherein the dihydropyridine compound is represented by formula II-a-1, II-b-1, II-c-1, or II-d-1:

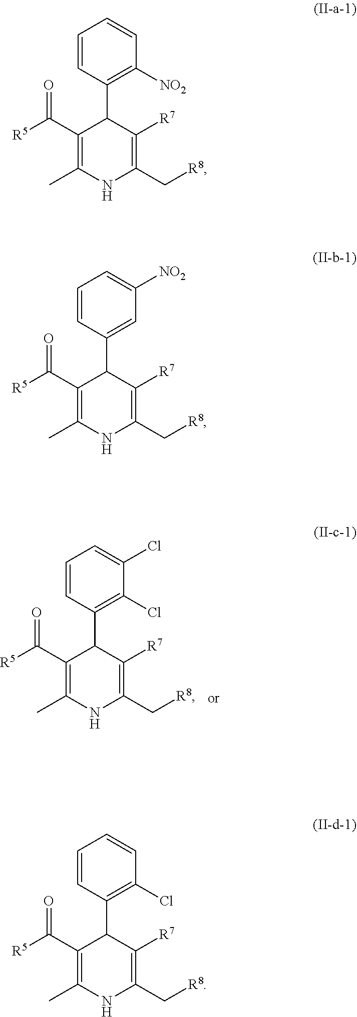

25. The kit of any one of claims 11-24, wherein R⁵ is alkoxy, amino, alkyl, carbocyclyl, heterocyclyl, aryl, or heteroaryl.

26. The kit of claim 25, wherein R⁵ is alkoxy or amino.

27. The kit of claim 26, wherein R⁵ is alkoxy optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl.

28. The kit of claim 27, wherein R⁵ is

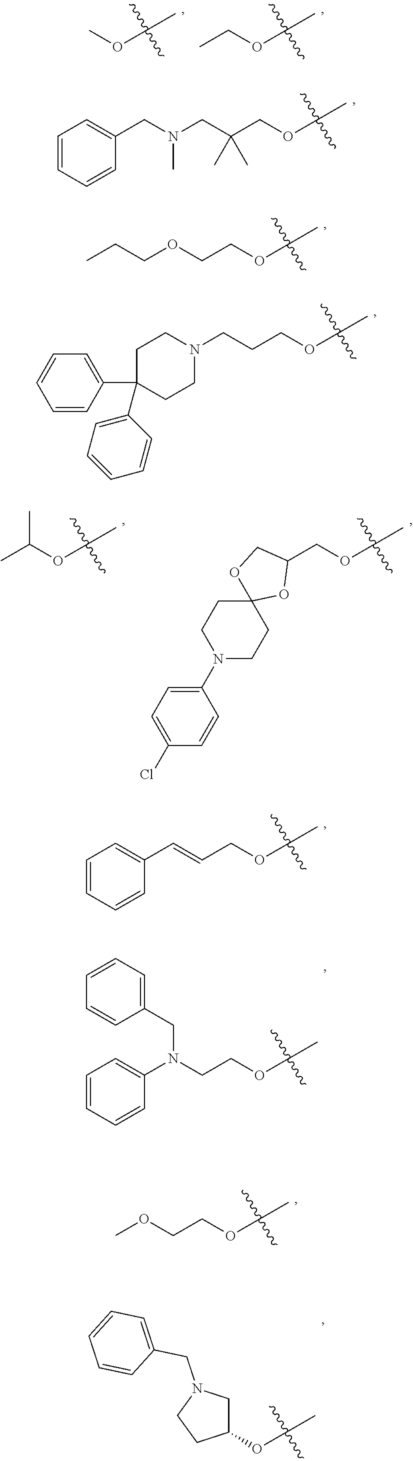

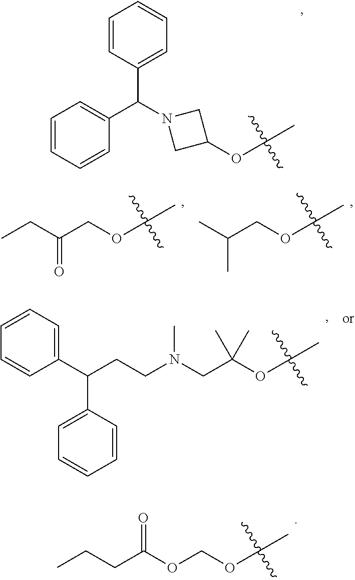

29. The kit of any one of claims 11-28, wherein R⁷ is acyl, ester, amide, alkyl, alkenyl, alkynyl, carbocyclyl, heterocyclyl, aryl, or heteroaryl.

30. The kit of claim 29, wherein R⁷ is acyl, ester, amide, carbocyclyl, heterocyclyl, aryl, or heteroaryl.

31. The kit of claim 30, wherein R⁷ is ester, heterocyclyl or heteroaryl.

32. The kit of claim 31, wherein R⁷ is

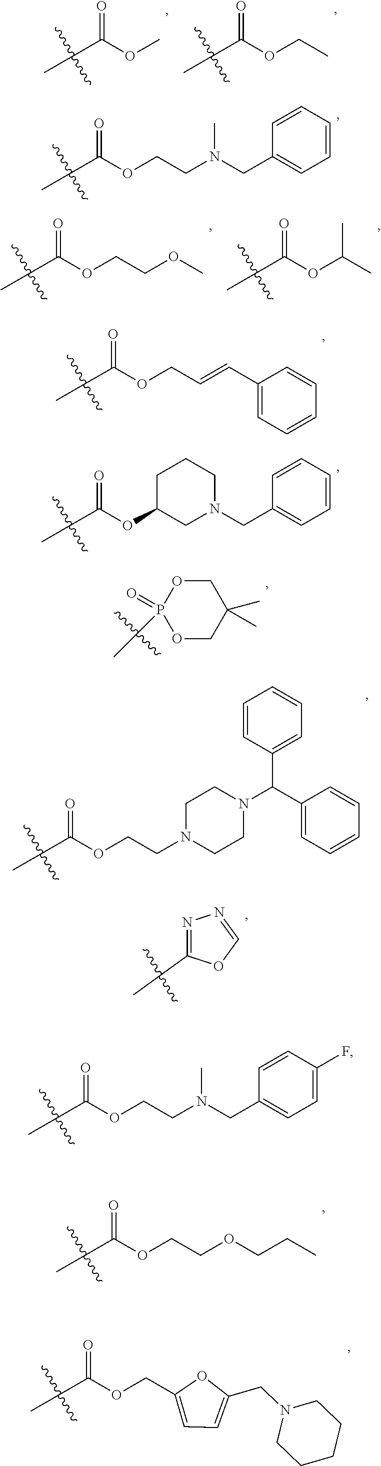

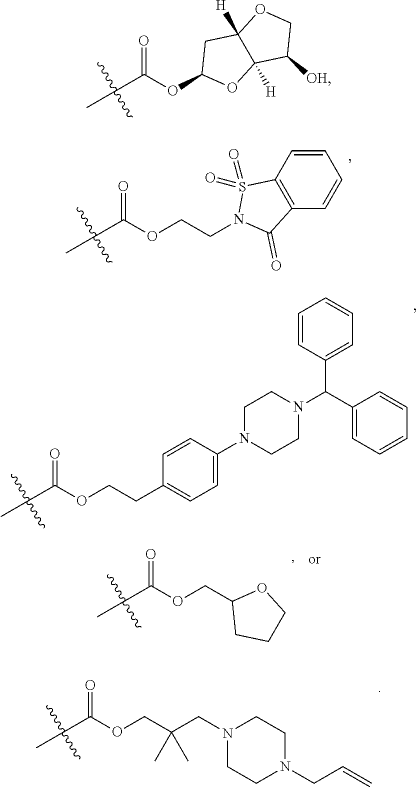 [SMILES: *C(=O)O[C@H]1C[C@@H]2OC[C@@H](O)[C@H]2O1 ; *C(=O)OCCN1C(=O)c2ccccc2S1(=O)=O ; *C(=O)OCCc1ccc(N2CCN(C(c3ccccc3)c3ccccc3)CC2)cc1 ; *C(=O)OCC1CCCO1 ; *C(=O)OCC(C)(C)CN1CCN(CC=C)CC1]

33. The kit of any one of claims 11-33, wherein R⁸ is hydrogen, hydroxyl, alkoxy, alkylthio, alkyl, alkenyl, alkynyl, carbocyclyl, heterocyclyl, aryl, or heteroaryl.

34. The kit of claim 33, wherein R⁸ is hydrogen, hydroxyl, alkoxy, alkylthio, or alkyl optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl.

35. The kit of claim 34, wherein R⁸ is hydrogen,

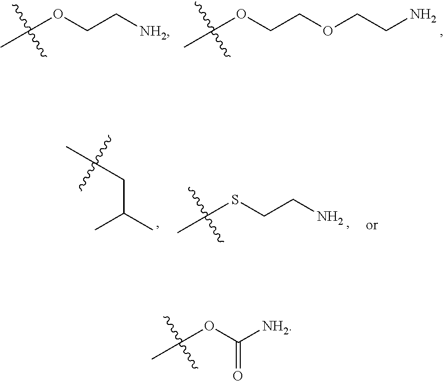

36. The kit of claim 10, wherein the dihydropyridine compound is selected from: 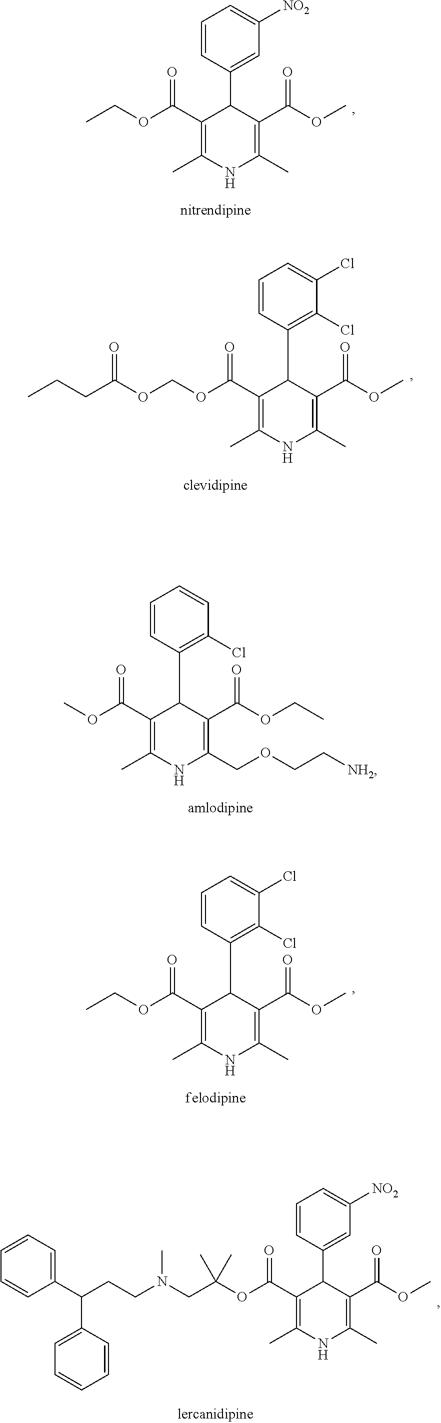 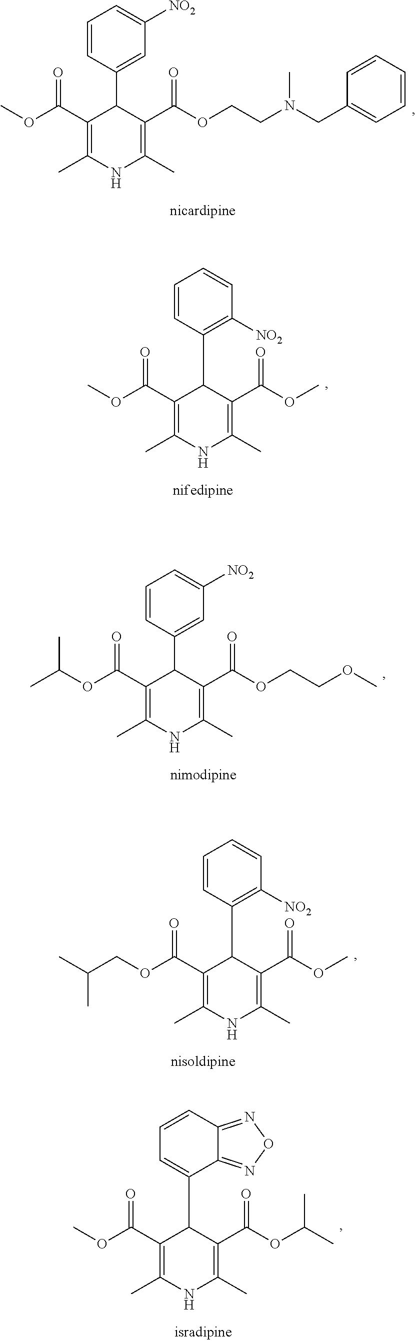 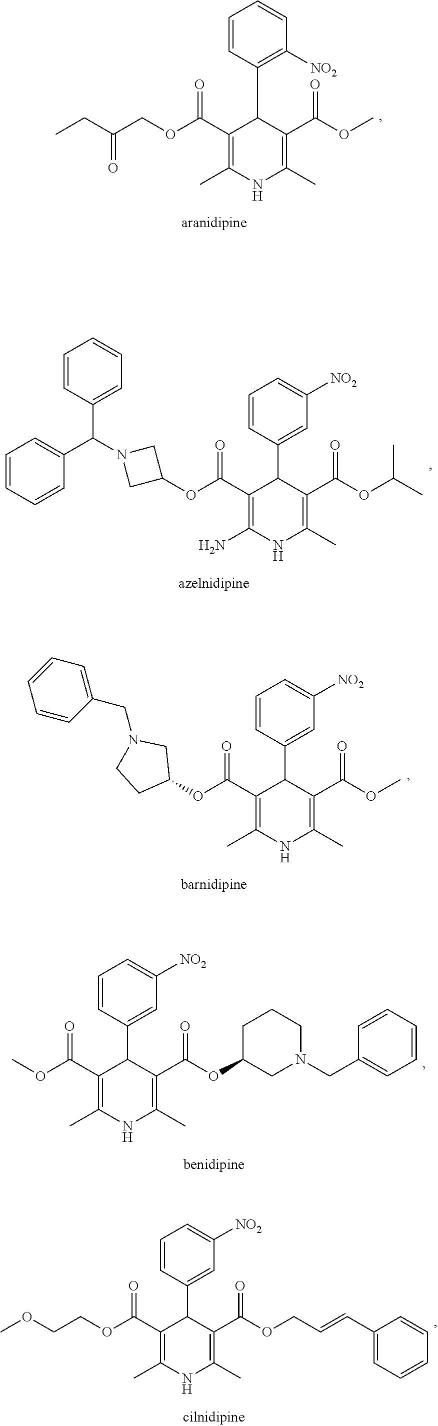 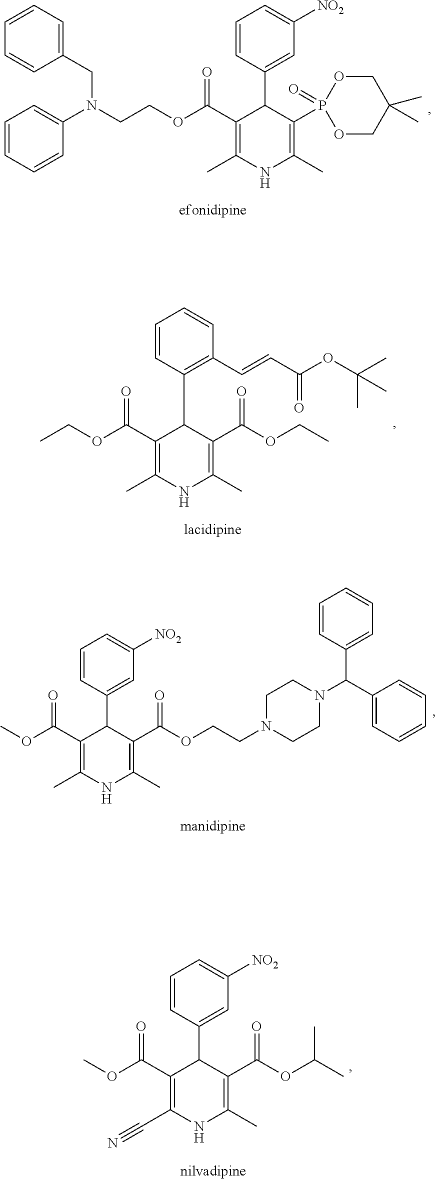 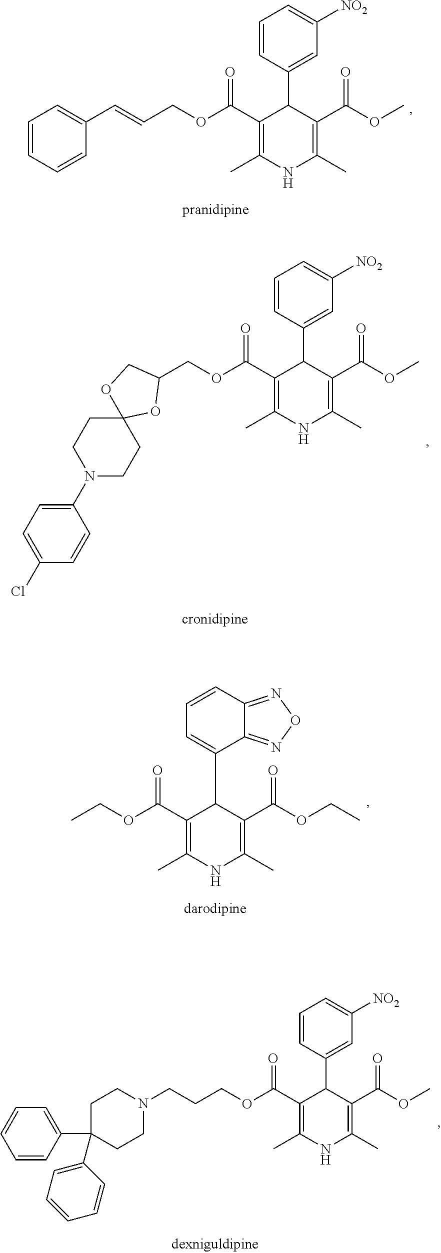 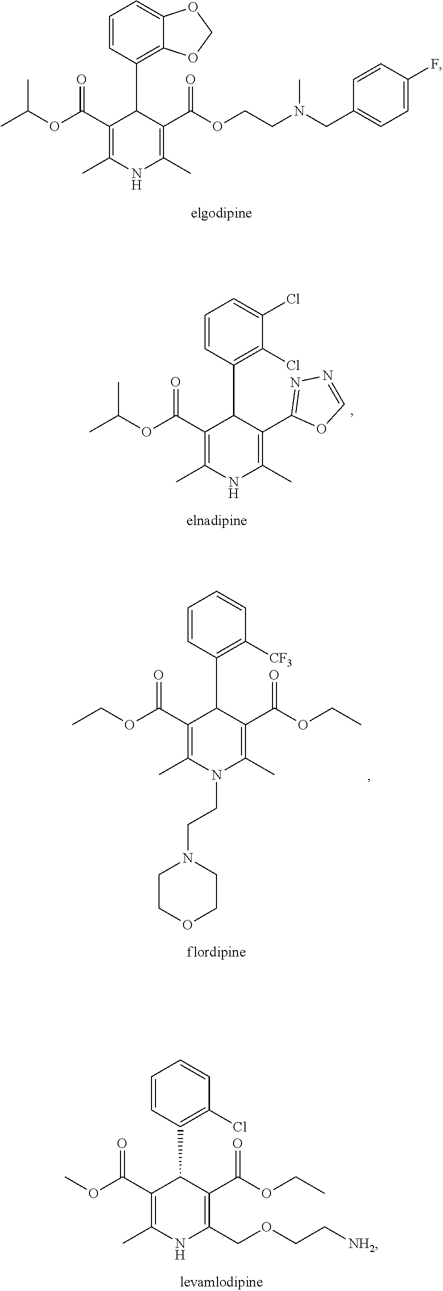 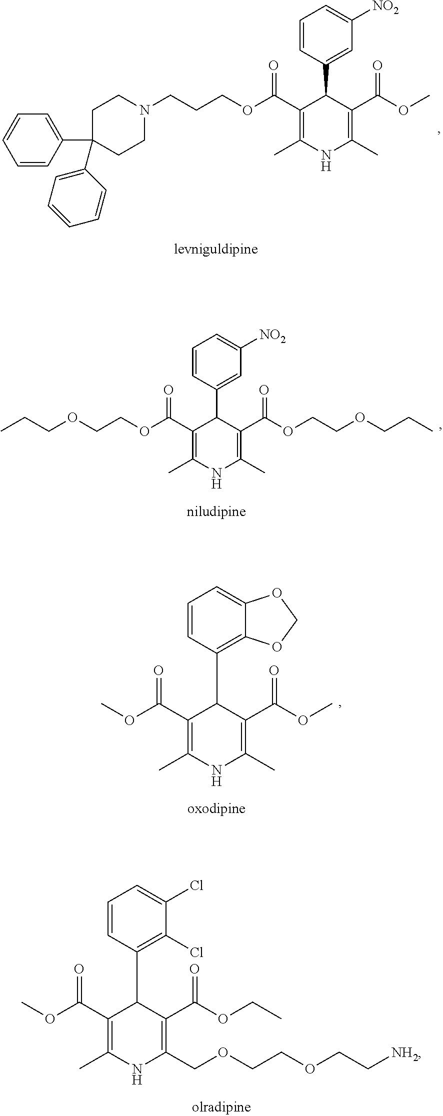 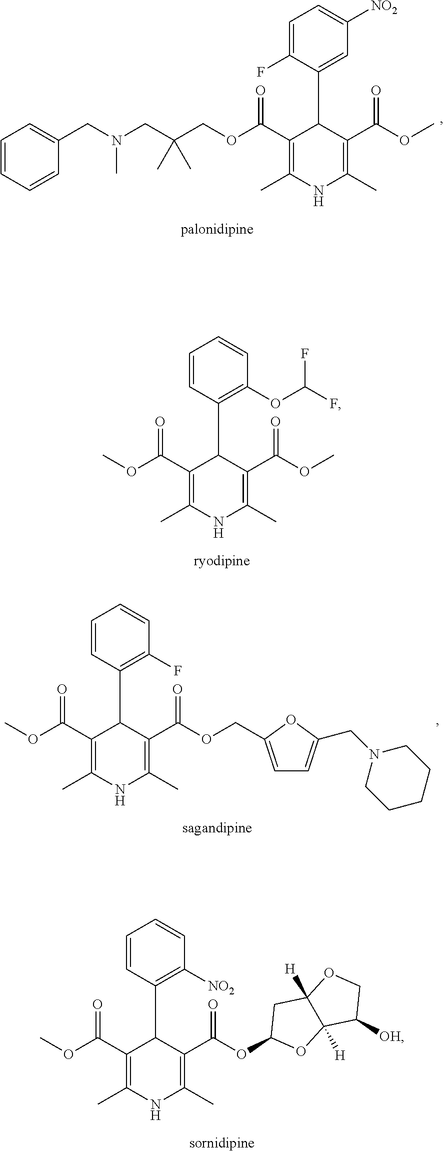 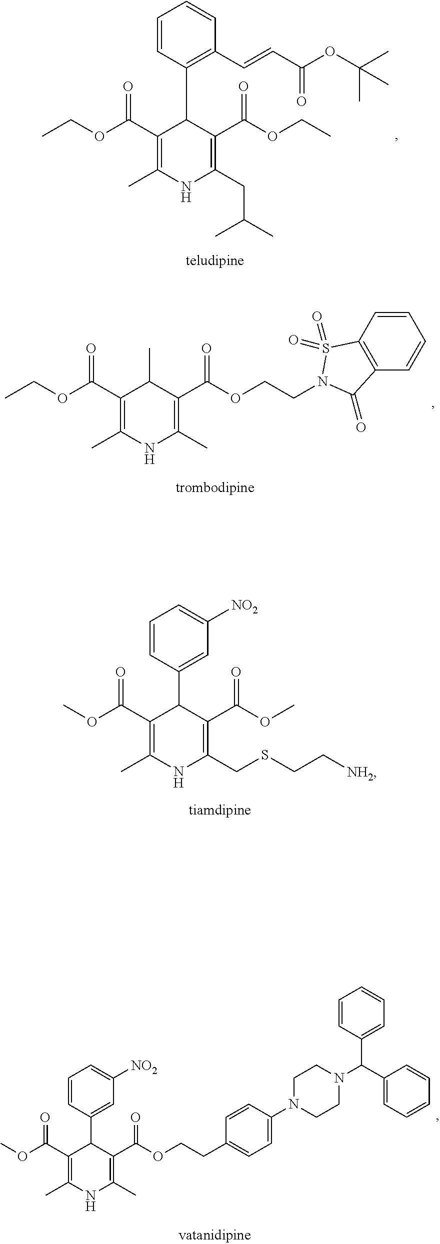 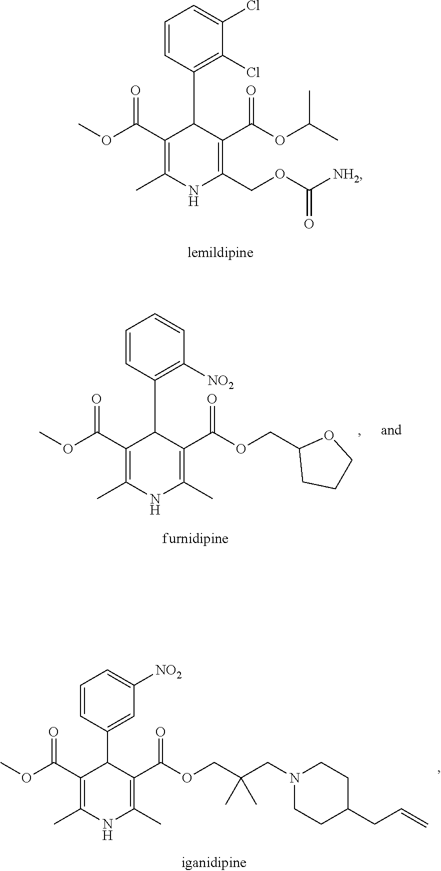 [SMILES: COC(=O)C1=C(C)NC(COC(N)=O)=C(C(=O)OC(C)C)C1c1cccc(Cl)c1Cl ; COC(=O)C1=C(C)NC(C)=C(C(=O)OCC2CCCO2)C1c1ccccc1[N+](=O)[O-] ; C=CCC1CCN(CC(C)(C)COC(=O)C2=C(C)NC(C)=C(C(=O)OC)C2c2cccc([N+](=O)[O-])c2)CC1]
or a pharmaceutically acceptable salt thereof.

37. The kit of claim 10, wherein the dihydropyridine compound is 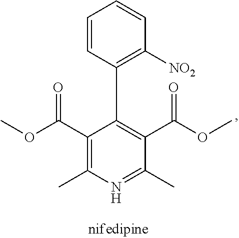
or a pharmaceutically acceptable salt thereof.

38. The kit of any one of claims 1-37, wherein the folate compound and the dihydropyridine compound are in the same composition.

39. A method of preventing or treating aneurysms, comprising conjointly administering a folate compound and a calcium channel blocker to a subject in need thereof.

40. A method of ameliorating a symptom of an aneurysm, comprising conjointly administering a folate compound and a calcium channel blocker to a subject in need thereof.

41. The method of claim 40, wherein the symptom is increased superoxide production, increased eNOS uncoupling activity, decreased nitric oxide (NO) bioavailability, decreased tetrahydrobiopterin (H₄B) bioavailability, enlargement of blood vessels (abdominal aortas, thoracic aortas or blood vessels in the brain), increased vascular remodeling, increased elastin degradation (flattening and breakdown), increased vascular inflammation/macrophage infiltration, increased matrix metalloproteinase (MMP) activation, increased adventitial hypertrophy, or a decrease in eNOS function.

42. A method of decreasing superoxide production, eNOS uncoupling activity, enlargement of blood vessels (abdominal aortas, thoracic aortas or blood vessels in the brain), vascular remodeling, elastin degradation (flattening and breakdown), vascular inflammation/macrophage infiltration, matrix metalloproteinase (MMP) activation, and/or adventitial hypertrophy in a subject afflicted with an aneurysm, comprising conjointly administering a folate compound and a calcium channel blocker to a subject in need thereof.

43. A method of increasing eNOS function, nitric oxide (NO), and tetrahydrobiopterin bioavailabilities in a subject afflicted with an aneurysm, the method comprising conjointly administering a folate compound and a calcium channel blocker to a subject in need thereof.

44. The method of any one of claims 39 to 43, wherein the folate compound is represented by formula I 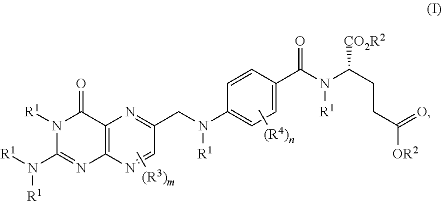
or a pharmaceutically acceptable salt thereof, wherein:
each R¹ independently is hydrogen, acyl, ester, amide, alkyl, alkenyl, alkynyl, carbocyclyl, heterocyclyl, aryl, or heteroaryl;
each R² independently is hydrogen, alkyl, alkenyl, alkynyl, carbocyclyl, heterocyclyl, aryl, or heteroaryl;
each R³ and R⁴ independently is halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl;
m is an integer selected from 0-3; and
n is an integer selected from 0-4.

45. The method of claim 44, wherein each R¹ independently is hydrogen, acyl, ester, amide, or alkyl.

46. The method of claim 44, wherein each R¹ is hydrogen.

47. The method of any one of claims 44 to 46, wherein each R² independently is hydrogen or alkyl.

48. The method of claim 47, wherein each R² is hydrogen.

49. The method of any one of claims 44 to 48, wherein m is 0.

50. The method of any one of claims 44 to 49, wherein n is 0.

51. The method of claim 44, wherein the folate compound is 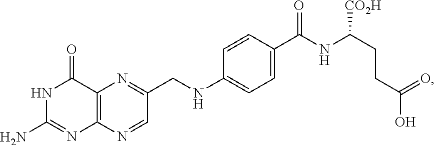 [SMILES: Nc1nc2ncc(CNc3ccc(C(=O)N[C@@H](CCC(=O)O)C(=O)O)cc3)nc2c(=O)[nH]1]
or a pharmaceutically acceptable salt thereof.

52. The method of any one of claims 39 to 43, wherein the calcium channel blocker is a dihydropyridine compound.

53. The method of claim 52, wherein the dihydropyridine compound is represented by formula II: 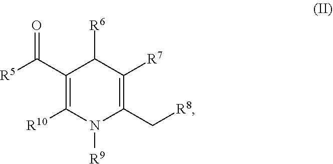
or a pharmaceutically acceptable salt thereof, wherein:
R⁵, R⁶, R⁷, R⁸, and R¹⁰ each independently is hydrogen, halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl; and
R⁹ is hydrogen, acyl, ester, amide, alkyl, alkenyl, alkynyl, carbocyclyl, heterocyclyl, aryl, or heteroaryl.

54. The method of claim 53, wherein R⁶ is alkyl, alkenyl, alkynyl, carbocyclyl, heterocyclyl, aryl, or heteroaryl.

55. The method of claim 54, wherein R⁶ is alkyl or aryl.

56. The method of claim 55, wherein R⁶ is methyl or substituted or unsubstituted phenyl.

57. The method of claim 56, wherein R⁶ is phenyl optionally substituted with halogen, haloalkyl, alkyl, or nitro.

58. The method of claim 53, wherein the dihydropyridine compound is represented by formula II-a, II-b, II-c, or II-d:

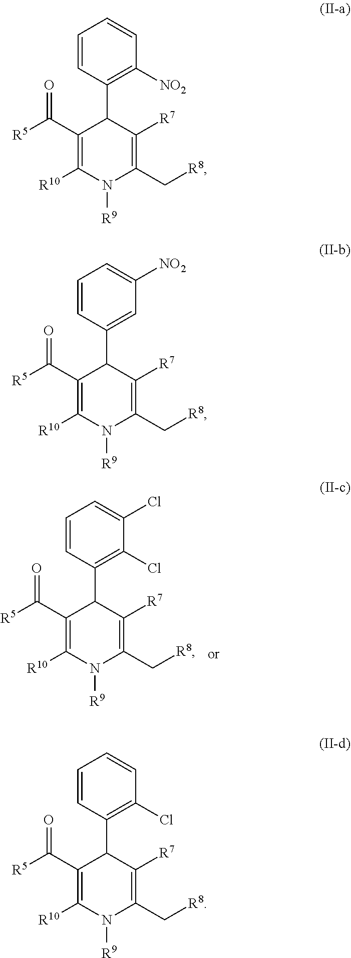

59. The method of any one of claims 53 to 58, wherein R⁹ is hydrogen, alkyl, alkenyl, alkynyl, carbocyclyl, heterocyclyl, aryl, or heteroaryl.

60. The method of claim 59, wherein R⁹ is hydrogen.

61. The method of claim 59, wherein R⁹ is alkyl optionally substituted with halogen, amino, hydroxyl, alkoxy, cyano, nitro, acyl, ester, amide, alkylthio, carbocyclyl, heterocyclyl, aryl, or heteroaryl.

62. The method of claim 61, wherein R⁹ is

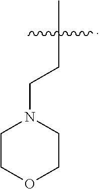 [SMILES: *CCN1CCOCC1]

63. The method of any one of claims 53 to 62, wherein R¹⁰ is cyano, amino, alkyl, alkenyl, alkynyl, carbocyclyl, heterocyclyl, aryl, or heteroaryl.

64. The method of claim 63, wherein R¹⁰ is cyano, amino, or alkyl.

65. The method of claim 64, wherein R¹⁰ is methyl.

66. The method of claim 53, wherein the dihydropyridine compound is represented by formula II-a-1, II-b-1, II-c-1, or II-d-1:

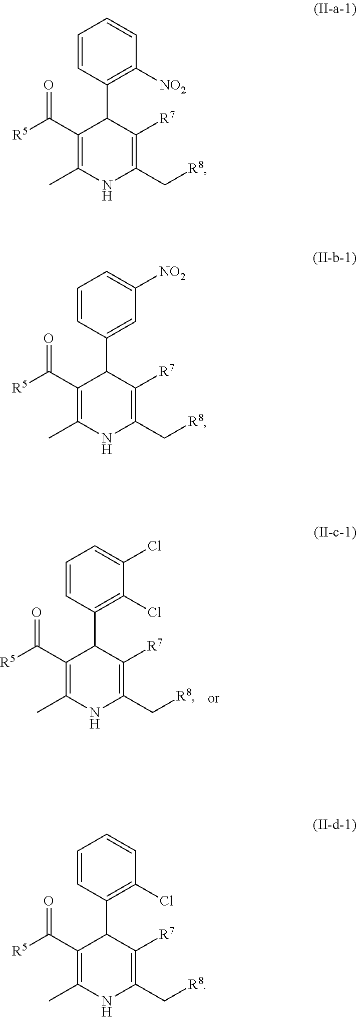

67. The method of any one of claims 53-66, wherein R⁵ is alkoxy, amino, alkyl, carbocyclyl, heterocyclyl, aryl, or heteroaryl.

68. The method of claim 67, wherein R⁵ is alkoxy or amino.

69. The method of claim 68, wherein R⁵ is alkoxy optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl.

70. The method of claim 69, wherein R⁵ is

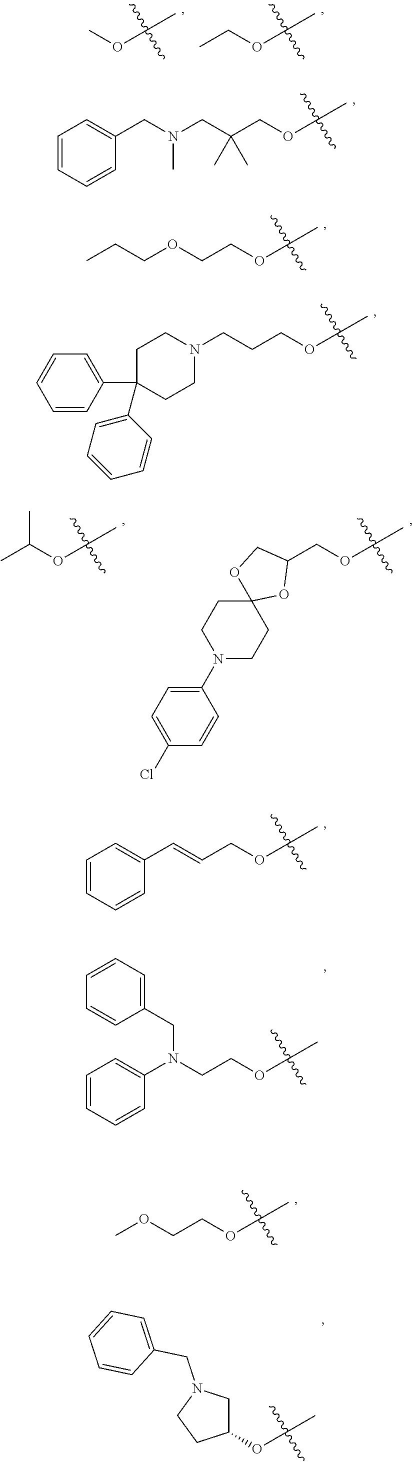

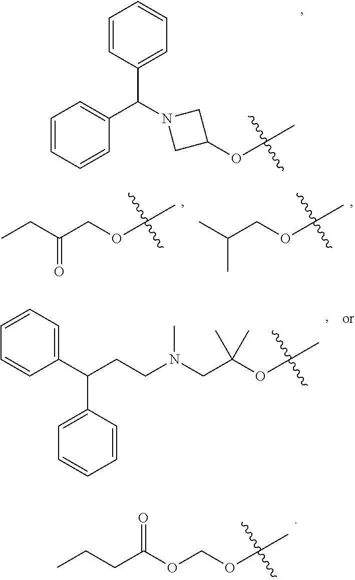

71. The method of any one of claims 53-70, wherein R⁷ is acyl, ester, amide, alkyl, alkenyl, alkynyl, carbocyclyl, heterocyclyl, aryl, or heteroaryl.

72. The method of claim 71, wherein R⁷ is acyl, ester, amide, carbocyclyl, heterocyclyl, aryl, or heteroaryl.

73. The method of claim 72, wherein R⁷ is ester, heterocyclyl or heteroaryl.

74. The method of claim 73, wherein R⁷ is

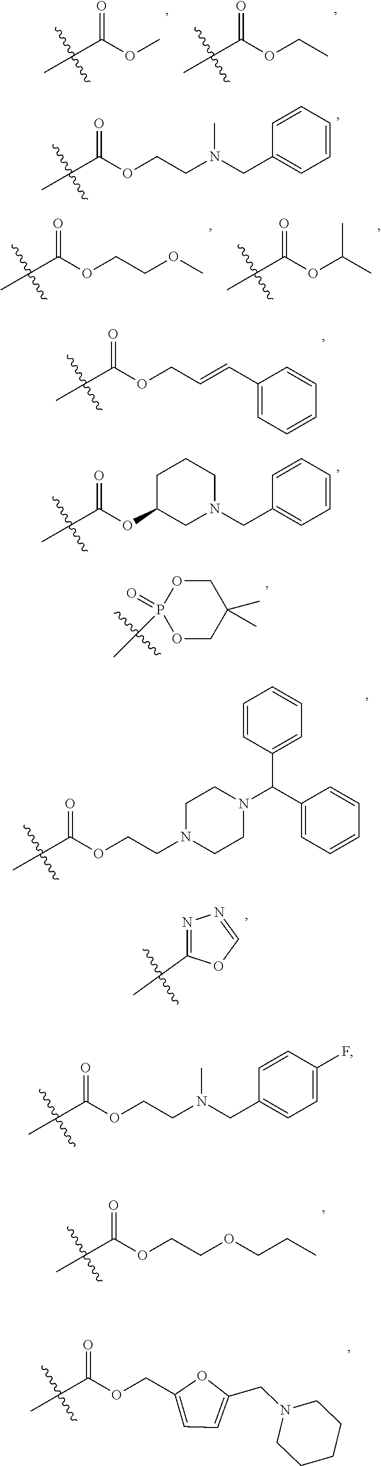

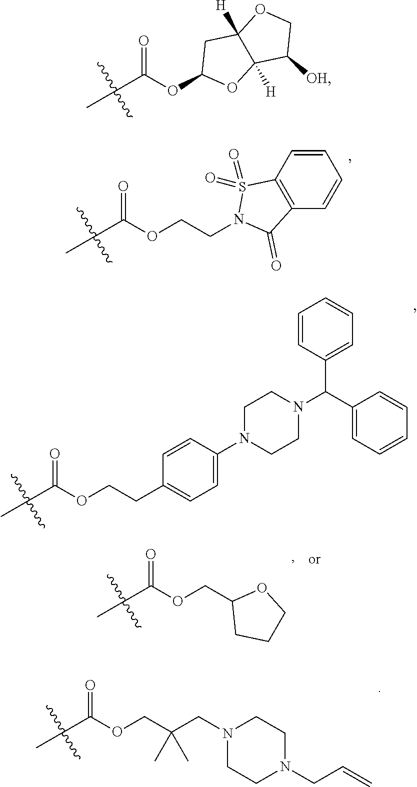 [SMILES: *C(=O)O[C@H]1C[C@@H]2OC[C@@H](O)[C@H]2O1 ; *C(=O)OCCN1C(=O)c2ccccc2S1(=O)=O ; *C(=O)OCCc1ccc(N2CCN(C(c3ccccc3)c3ccccc3)CC2)cc1 ; *C(=O)OCC1CCCO1 ; *C(=O)OCC(C)(C)CN1CCN(CC=C)CC1]

75. The method of any one of claims 53-74, wherein R⁸ is hydrogen, hydroxyl, alkoxy, alkylthio, alkyl, alkenyl, alkynyl, carbocyclyl, heterocyclyl, aryl, or heteroaryl.

76. The method of claim 75, wherein R⁸ is hydrogen, hydroxyl, alkoxy, alkylthio, or alkyl optionally substituted with halogen, cyano, nitro, amino, hydroxyl, alkylthio, alkoxy, acyloxy, acylamino, acyl, ester, amido, alkyl, alkenyl, alkynyl, carbocyclyl, heterocyclyl, aryl, or heteroaryl.

77. The method of claim 76, wherein R⁸ is hydrogen,

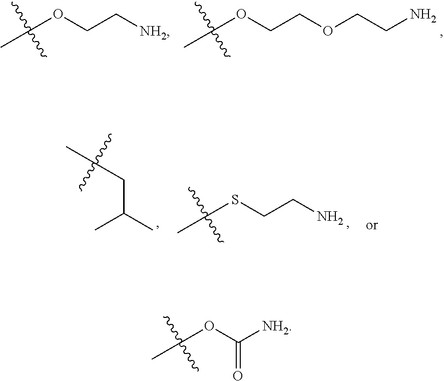

78. The method of claim 52, wherein the dihydropyridine compound is selected from: 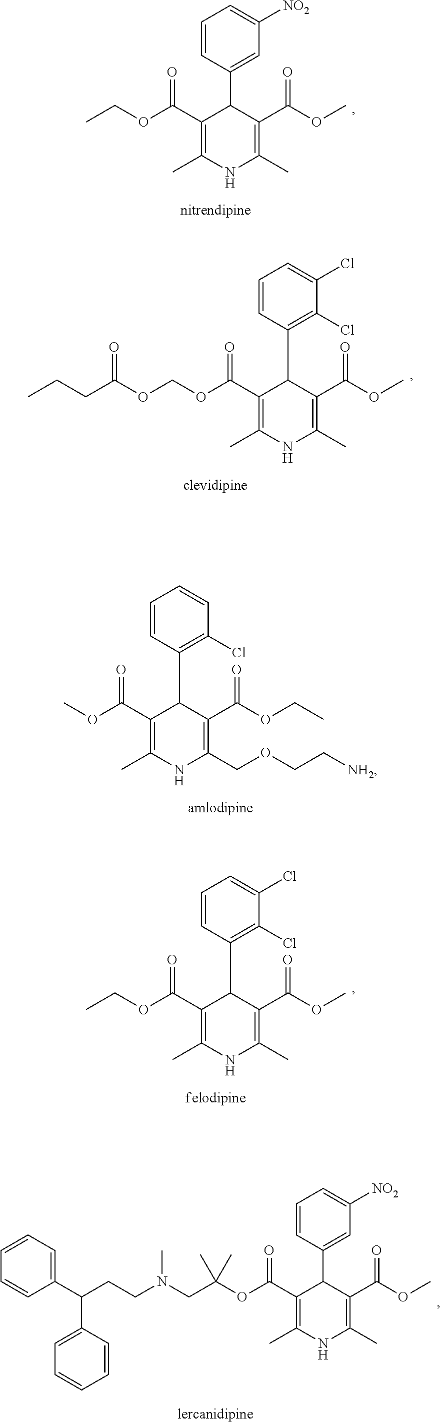 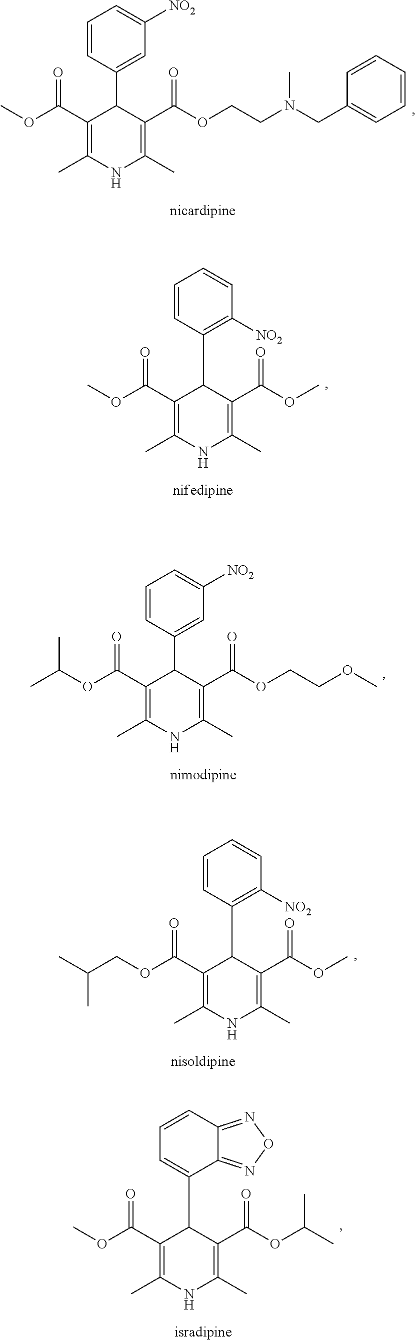 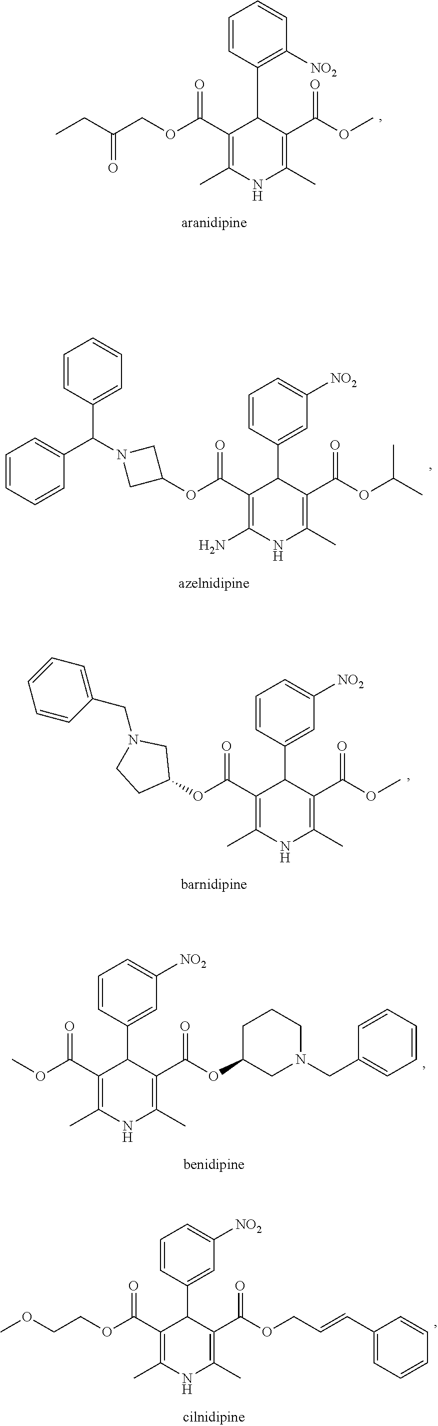 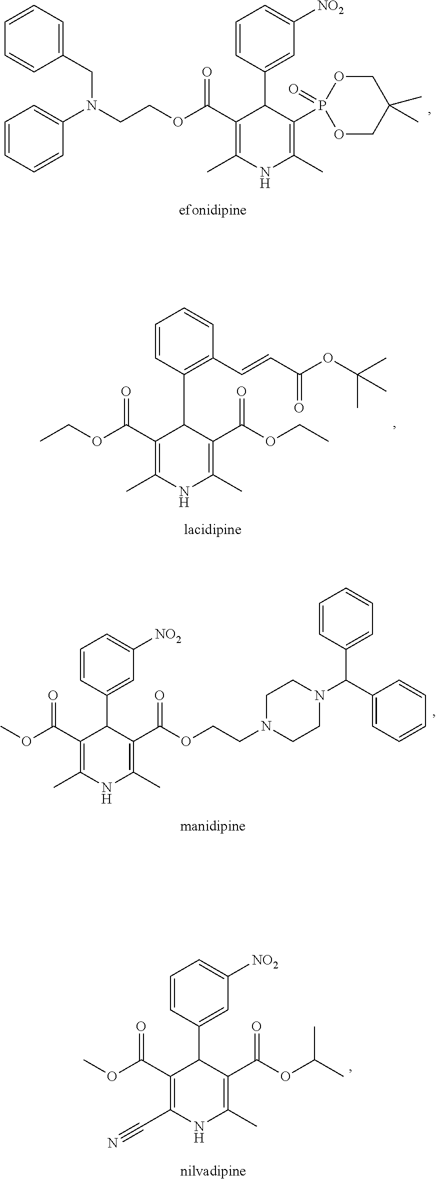 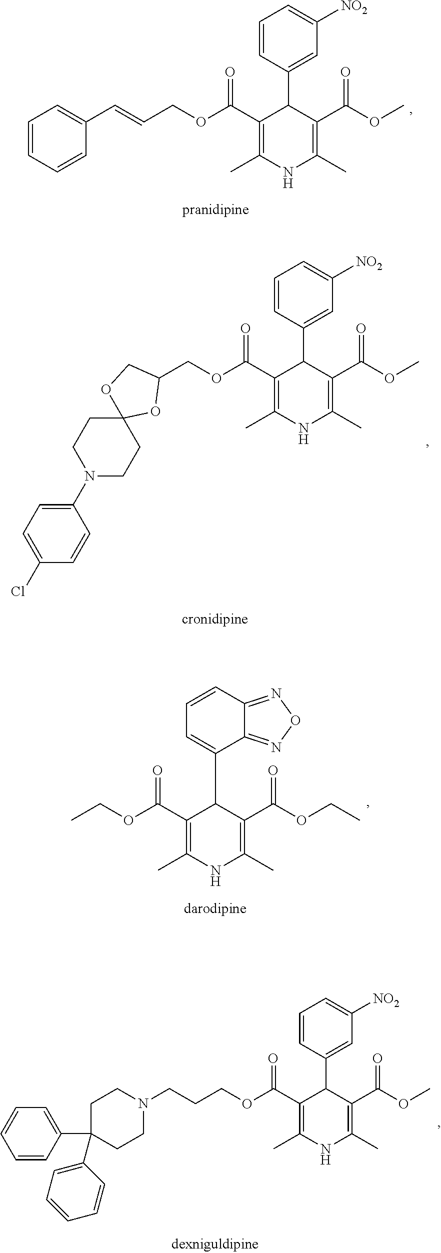 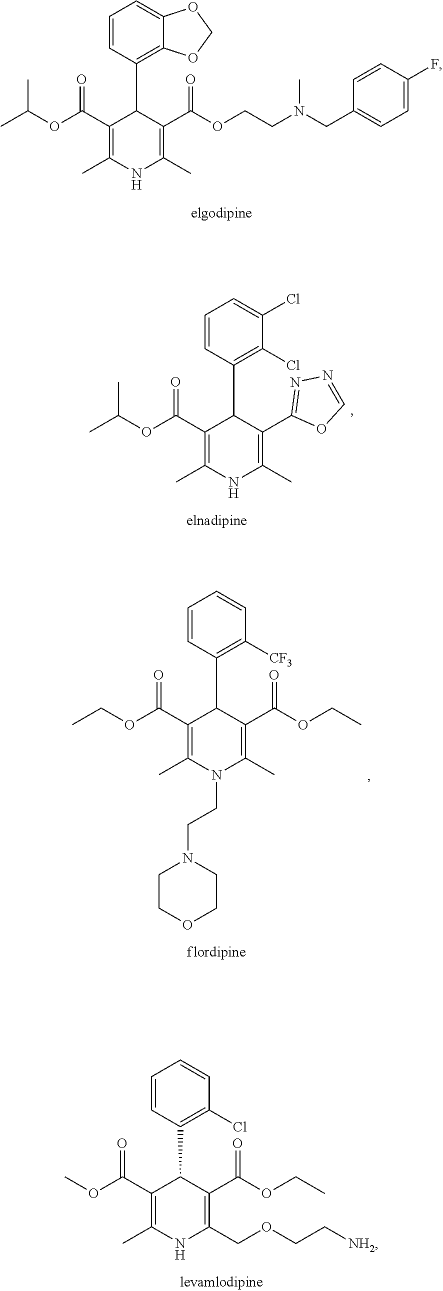 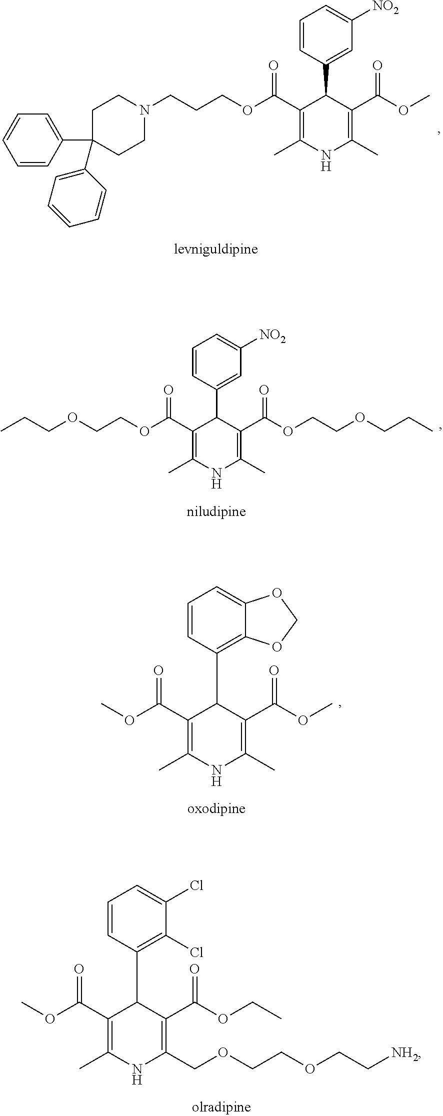 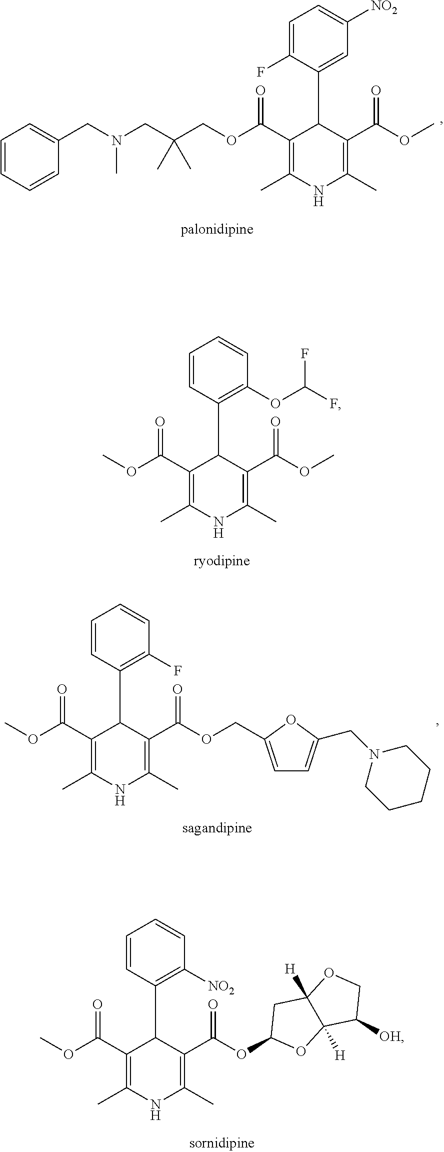 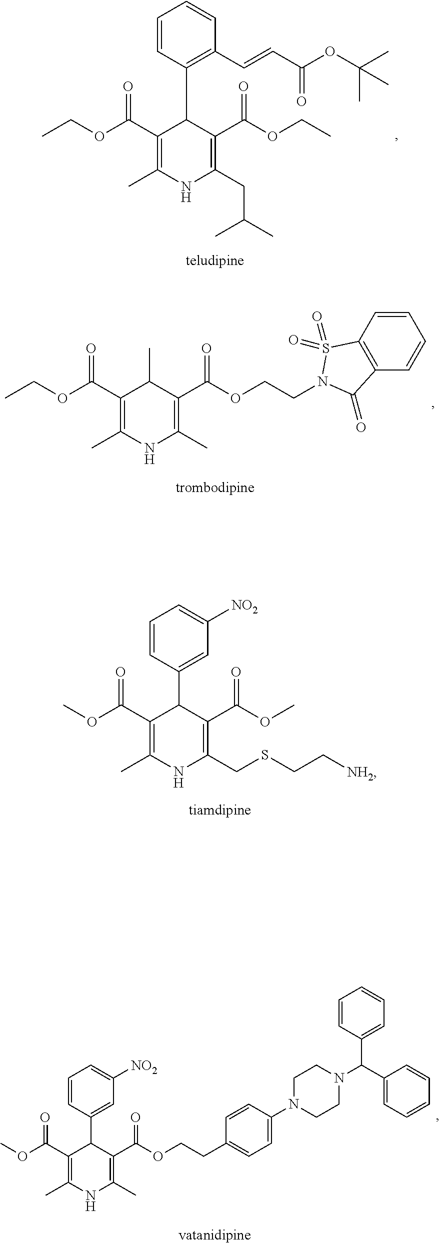 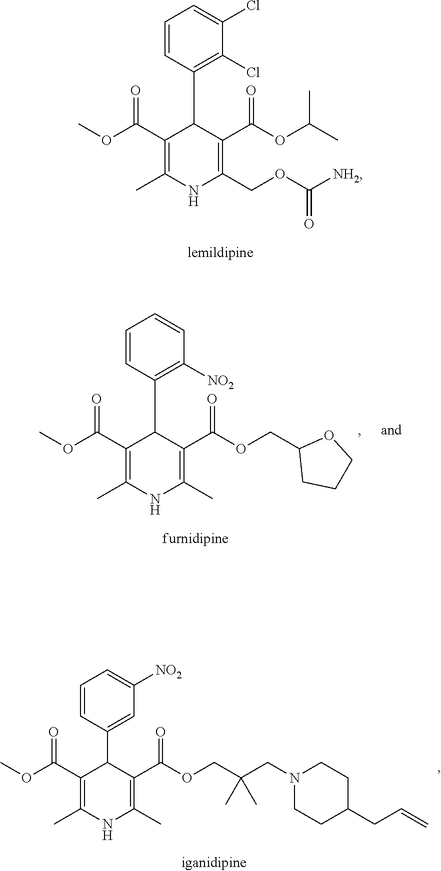 [SMILES: COC(=O)C1=C(C)NC(COC(N)=O)=C(C(=O)OC(C)C)C1c1cccc(Cl)c1Cl ; COC(=O)C1=C(C)NC(C)=C(C(=O)OCC2CCCO2)C1c1ccccc1[N+](=O)[O-] ; C=CCC1CCN(CC(C)(C)COC(=O)C2=C(C)NC(C)=C(C(=O)OC)C2c2cccc([N+](=O)[O-])c2)CC1]
or a pharmaceutically acceptable salt thereof.

79. The method of claim 52, wherein the dihydropyridine compound is 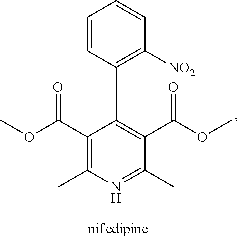
or a pharmaceutically acceptable salt thereof.

80. The method of any one of claims 52-79, wherein the folate compound and the dihydropyridine compound are in the same composition.

81. The method of any one of claims 39-80, wherein the folate compound is folic acid.

82. The method of any one of claims 39-81, wherein the calcium channel blocker is Nifedipine.

83. The method of any one of claims 39-78, wherein the folate compound and the calcium channel blocker are administered in a therapeutically effective amount.

84. The method of any one of claims 39-83, wherein the folate compound and the calcium channel blocker are administered in a mass ratio of about 10:1 to about 1:10.

85. The method of claim 84, wherein the folate compound and the calcium channel blocker are administered in a mass ratio of about 3:1 to about 3:4.

86. The method of any one of claims 39-85, wherein the folate compound is administered in an amount of about 1-350 mg, about 1-700 mg, about 1-1050 mg, about 1-1400 mg, or about 1-1750 mg.

87. The method of any one of claims 39-86, wherein the calcium channel block is administered in an amount of about 1-350 mg, about 1-700 mg, about 1-1050 mg, about 1-1400 mg, about 1-1750 mg, about 1-2100 mg, or about 1-2450 mg.

88. The method of any one of claims 39-87, wherein the folate compound and the calcium channel blocker are administered simultaneously.

89. The method of any one of claims 39-88, wherein the folate compound and the calcium channel blocker are administered sequentially.

90. The method of claim 89, wherein the folate compound and the calcium channel blocker are in separate dosage forms.

91. The method of any one of claims 39-90, wherein the aneurysm is abdominal aortic aneurysm, cerebral aneurysm, or thoracic aortic aneurysm.

92. The method of any one of claims 39-91, wherein the folate compound and the calcium channel blocker are administered orally.
